(19) United States
(12) Patent Application Publication
(10) Pub. No.: US 2022/0081385 A1
(43) Pub. Date: Mar. 17, 2022

(54) NOVEL SALTS AND POLYMORPHIC FORM OF BEMPEDOIC ACID

(71) Applicant: Lupin Limited, Mumbai (IN)

(72) Inventors: Navnath Ambadas Kadam, Pune (IN); Rohidas Shivaji Sulake, Pune (IN); Rajinder Singh Siyan, Pune (IN); Nandu Baban Bhise, Pune (IN); Girij Pal Singh, Pune (IN); Rajesh Harishankar Vyas, Pune (IN)

(21) Appl. No.: 17/419,939

(22) PCT Filed: Dec. 27, 2019
(86) PCT No.: PCT/IB2019/061391

§ 371 Date: Jun. 30, 2021

(30) Foreign Application Priority Data
Dec. 31, 2018 (IN) 201821049982
Jul. 3, 2019 (IN) 201921026733

(51) Int. Cl. C07C 67/313

(52) U.S. Cl. CPC C07C 67/313

(57) ABSTRACT

The present invention relates to novel pharmaceutically acceptable salts of Bempedoic acid, novel intermediates of Bempedoic acid, novel crystalline form of Bempedoic acid and novel processes for the preparation of Bempedoic acid or its intermediates thereof.

This application claims priority and benefit of following Indian provisional patent application no. 201821049982, filed on Dec. 31, 2018 and Indian provisional patent application no. 201921026733, filed on Jul. 3, 2019.

### FIELD OF THE INVENTION

The present invention relates to novel pharmaceutically acceptable salts of Bempedoic acid and process for the preparation thereof.

The present invention also relates to novel Bempedoic acid intermediates and processes for the preparation thereof.

The present invention also relates to novel crystalline form of Bempedoic acid and process for the preparation thereof.

The present invention further relates to novel processes for the preparation of Bempedoic acid.

### BACKGROUND OF THE INVENTION

Bempedoic acid is chemically known as 8-hydroxy-2, 2, 14, 14-tetramethylpentadecanedioic acid and its chemical structure is depicted below in formula (I).

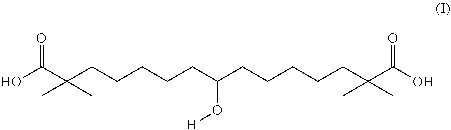 [SMILES: CC(C)(CCCCCC(O)CCCCCC(C)(C)C(=O)O)C(=O)O]

Bempedoic acid is useful in the treatment of hypercholesterolemia and hypertension.

The U.S. Pat. No. 7,335,799 describes preparation of Bempedoic acid by using 8-oxo-2,2,14,14-tetramethyl-pentadecanedioic acid, Bempedoic acid was isolated as viscous oil in example number 6.20. US '799 patent does not disclose the solid-state crystalline properties of Bempedoic acid.

Present invention relates to novel pharmaceutically acceptable salts of Bempedoic acid, novel Bempedoic acid intermediates, novel crystalline polymorphic form of Bempedoic acid and processes for the preparation thereof.

### SUMMARY OF THE INVENTION

The present invention relates to novel pharmaceutically acceptable organic and inorganic salts of Bempedoic acid and process for the preparation thereof.

The Present invention also relates to novel Bempedoic acid intermediates and processes for the preparation thereof.

The present invention further relates to novel processes for the preparation of Bempedoic acid.

The present invention also relates to crystalline form of Bempedoic acid and process for the preparation thereof.

### DESCRIPTION OF THE DRAWINGS

FIG. 1 is an illustration of a powder X-ray diffraction (PXRD) pattern of solid crystalline form of Bempedoic acid described in the present invention.

FIG. 2 is an illustration of a differential scanning calorimetric profile of solid crystalline form of Bempedoic acid described in the present invention.

### DETAILED DESCRIPTION OF THE INVENTION

One aspect of the present invention provides pharmaceutically acceptable salts of Bempedoic acid or it solvates or hydrates thereof and process for the preparation thereof.

Another aspect of the present invention provides pharmaceutically acceptable salt of Bempedoic acid include salts with alkaline metals (like, lithium, sodium, potassium, etc.), alkaline earth metals (like, magnesium, calcium, barium, etc.), transition metals (like, zinc, iron, etc.). Further, organic bases (like, trimethylamine, triethylamine, dicyclohexylamine, ethanolamine, diethanolamine, triethanolamine, piperazine, tert-buty amine, meglumine, ethylenediamine, pyridine, picoline, quinolin, etc.), amino acids, or mixtures thereof. These salts prepared in accordance with the conventional methods.

Yet another aspect of the present invention provides sodium salt of Bempedoic acid or its hydrate and solvates thereof.

Another aspect of the present invention provides sodium salt of Bempedoic acid (compound of formula AA).

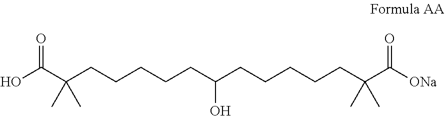

Yet another aspect of the present invention provides a process for the preparation of sodium salt of Bempedoic acid comprising the steps of:
a) Bempedoic acid is treating with solvent,
b) adding a base, selected from sodium containing base, and
c) isolating sodium salt of Bempedoic acid.

Another aspect of the present invention provides potassium salt of Bempedoic acid or its hydrate and solvates thereof.

Yet another aspect of the present invention provides a process for the preparation of potassium salt of Bempedoic acid (compound of formula BB).

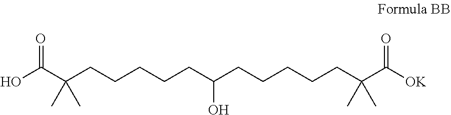

Another aspect of the present invention provides a process for the preparation of potassium salt of Bempedoic acid comprising the steps of:
i. Bempedoic acid is treating with solvent,
ii. adding a base, selected from potassium containing base, and
iii. isolating potassium salt of Bempedoic acid.

Yet another aspect of the present invention provides calcium salt of Bempedoic acid or it hydrate and solvates thereof.

Another aspect of the present invention provides a process for the preparation of calcium salt of Bempedoic acid (compound of formula CC).

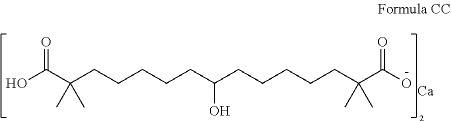

Yet another aspect of the present invention provides a process for the preparation of calcium salt of Bempedoic acid comprising the steps of:
i. Bempedoic acid is treating with solvent,
ii. adding a base, optionally selected from sodium hydroxide,
iii. adding calcium acetate and water to step ii, and
iv. isolating calcium salt of Bempedoic acid.

Another aspect of the present invention provides piperazine salt of Bempedoic acid or its hydrate and solvates thereof.

Another aspect of the present invention provides a process for the preparation of piperazine salt of Bempedoic acid (Compound of formula DD).

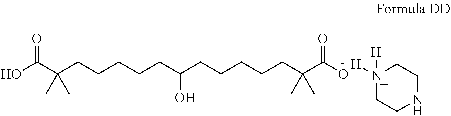

Yet another aspect of the present invention provides a process for the preparation of piperazine salt of Bempedoic acid comprising the steps of:
a) Bempedoic acid is treating with solvent,
b) adding piperazine solution, and
c) isolating piperazine salt of Bempedoic acid.

Another aspect of the present invention provides bis-piperazine salt of Bempedoic acid or its hydrate and solvates thereof.

Yet another aspect of the present invention provides a process for the preparation of bis-piperazine salt of Bempedoic acid (Compound of formula EE).

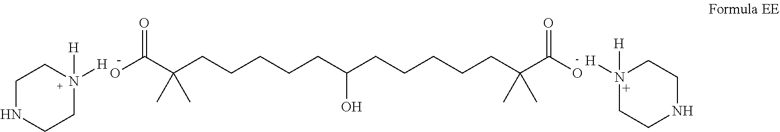

Another aspect of the present invention provides a process for the preparation of bis-piperazine salt of Bempedoic acid comprising the steps of:
a) Bempedoic acid is treating with solvent,
b) adding piperazine, optionally heating and
c) isolating bis-piperazine salt of Bempedoic acid.

Yet another aspect of the present invention provides bis-tert-butyl salt of Bempedoic acid its hydrate and solvates thereof.

Another aspect of the present invention provides a process for the preparation of bis-tert-butyl salt of Bempedoic acid (Compound of formula FF).

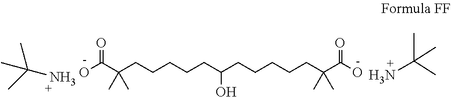

Yet another aspect of the present invention provides a process for the preparation of bis-tert-butyl salt of Bempedoic acid comprising the steps of:
a) Bempedoic acid is treating with solvent,
b) adding tert-butyl amine,
c) isolating bis-tert butyl amine salt of Bempedoic acid.

According to the process of the present invention, pharmaceutically acceptable salt of Bempedoic acid may form a solvate, such as hydrate, and/or a crystalline polymorph or amorphous. The present invention includes such various solvates as well as polymorphs. “Solvates” may be those wherein any numbers of solvent molecules (like methanol, ethanol, 1-propanol, 2-propanol, 1-butanol, isobutanol, tert-butanol, 2-methoxyethanol, 2,2,2-trifluoroethanol; or acetonitrile, nitromethane, 1,2-dimethoxyethane; or esters, such as methyl acetate, ethyl acetate, or ketones, such as e.g. acetone, 2-butanone; or mixtures thereof, or mixtures with water) are coordinated with the compound of present the invention. When the compound of the present invention or a pharmaceutically acceptable salt thereof is allow standing in the atmosphere, it may absorb water, resulting in attachment of adsorbed water or formation of hydrates.

According to the process of the present invention, sodium salt of Bempedoic acid, potassium salt of Bempedoic acid, calcium salt of Bempedoic acid, piperazine salt of Bempedoic acid, bis-piperazine salt of Bempedoic acid, bis-tert-butyl salt of Bempedoic acid is prepared with high purity.

According to the present invention, solvent is selected from alcohol such as methanol, ethanol, isopropanol, n-propanol, tertiary-butyl alcohol; ketone solvents such as acetone, methyl isobutyl ketone, ethyl methyl ketone; chlorinated solvents such as dichloromethane, chloroform, carbon tetrachloride; esters such as methyl acetate, ethyl acetate, n-propyl acetate, n-butyl acetate, t-butyl acetate; ether solvents such as tetrahydrofuran, 2-methyl tetrahydrofuran, dimethyl ether, diethyl ether, diisopropyl ether, methyl tert-butyl ether; nitriles such as acetonitrile, butyronitrile, isobutyronitrile, polar aprotic solvents such as dimethyl acetamide, dimethylsulfoxide, dimethylformamide, N-methyl-2-pyrrolidone, water or a mixture thereof.

Yet another aspect of the present invention provides novel Bempedoic acid intermediates and processes for the preparation thereof.

Another aspect of the present invention provides novel process for the preparation of Bempedoic acid of formula I by using any one of the novel Bempedoic acid intermediates selected from compound of formula 2, compound of formula 3, compound of formula 4, compound of formula 5, compound of formula 6, compound of formula 7, compound of formula XA, compound of formula XB, and compound of formula XC.

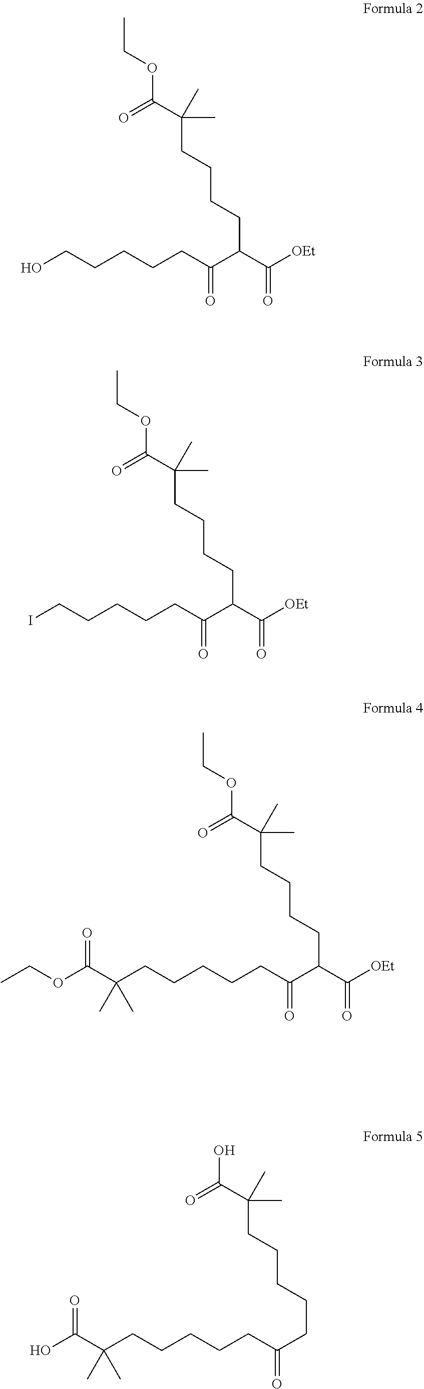 [SMILES: CCOC(=O)C(CCCCC(C)(C)C(=O)OCC)C(=O)CCCCCO ; CCOC(=O)C(CCCCC(C)(C)C(=O)OCC)C(=O)CCCCCI ; CCOC(=O)C(CCCCC(C)(C)C(=O)OCC)C(=O)CCCCCC(C)(C)C(=O)OCC ; CC(C)(CCCCCC(=O)CCCCCC(C)(C)C(=O)O)C(=O)O]

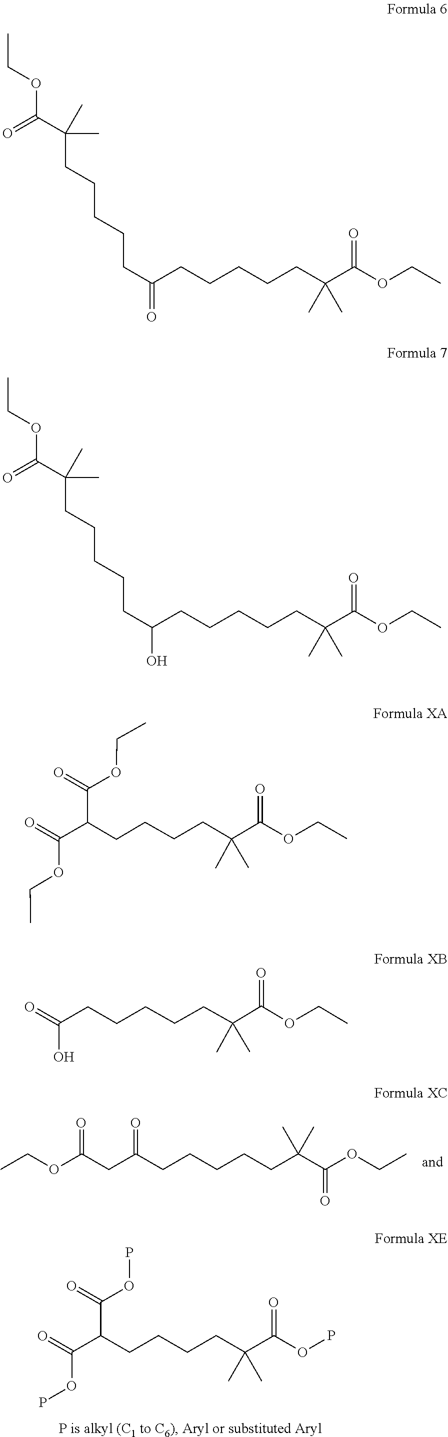

Yet another aspect of the present invention provides crystalline form of Bempedoic acid and process for the preparation thereof.

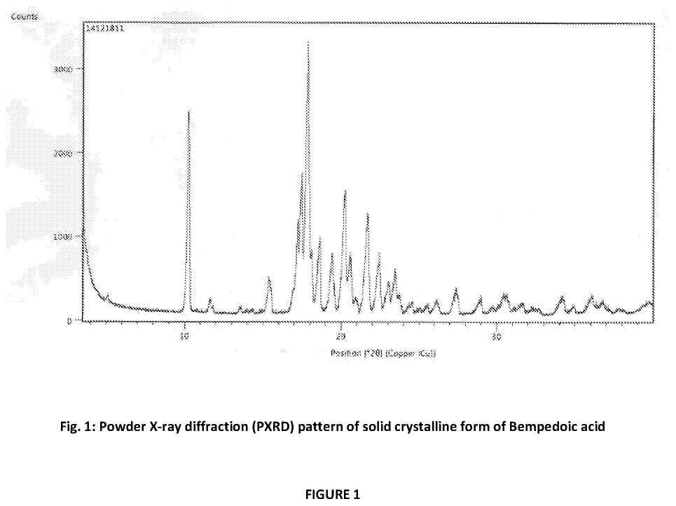

A powder X-ray powder diffraction pattern as depicted in FIG. 1 characterizes the crystalline form of Bempedoic acid of the present invention.

Yet another aspect of the present invention crystalline form of Bempedoic acid having PXRD characteristic peaks at 10.2°±0.2°, 17.4°±0.2°, 17.8°±0.2°, 18.6°±0.2°, 20.2°±0.2°, 21.7°±0.2°, 22.4°±0.2° and 23.4°±0.2° degrees 2θ.

Another aspect of the present invention crystalline form of Bempedoic acid having PXRD characteristic peaks, d-spacing and relative intensity shown in below Table-1.

TABLE 1

| 2 theta | D spacing | Relative intensity |
|---|---|---|
| 5.07 | 17.41 | 1.44 |
| 10.2 | 8.61 | 60.56 |
| 11.6 | 7.59 | 6.02 |
| 13.5 | 6.52 | 2.26 |
| 14.0 | 6.31 | 1.11 |
| 14.3 | 6.16 | 1.65 |
| 15.5 | 5.69 | 9.10 |
| 17.2 | 5.14 | 37.31 |
| 17.3 | 5.09 | 54.36 |
| 17.4 | 5.07 | 59.19 |
| 17.8 | 4.96 | 100 |
| 18.1 | 4.88 | 24.36 |
| 18.6 | 4.75 | 29.05 |
| 19.4 | 4.57 | 26.86 |
| 20.2 | 4.37 | 46.58 |
| 20.6 | 4.29 | 15.43 |
| 20.9 | 4.23 | 5.88 |
| 21.7 | 4.09 | 37.41 |
| 22.4 | 3.95 | 20.44 |
| 23.0 | 3.85 | 12.67 |
| 23.4 | 3.78 | 17.22 |
| 23.7 | 3.73 | 7.07 |
| 24.3 | 3.65 | 3.61 |
| 24.5 | 3.62 | 3.49 |
| 25.0 | 3.55 | 2.43 |
| 25.6 | 3.47 | 2.59 |
| 26.1 | 3.40 | 5.12 |
| 27.4 | 3.24 | 9.27 |
| 28.9 | 3.07 | 6.03 |
| 29.7 | 3.00 | 3.38 |
| 30.4 | 2.93 | 8.34 |
| 31.6 | 2.82 | 4.22 |
| 32.3 | 2.76 | 2.35 |
| 32.3 | 2.73 | 2.11 |
| 34.3 | 2.61 | 5.30 |
| 34.9 | 2.56 | 2.57 |
| 36.0 | 2.48 | 7.20 |
| 36.8 | 2.44 | 4.80 |
| 37.7 | 2.38 | 1.50 |

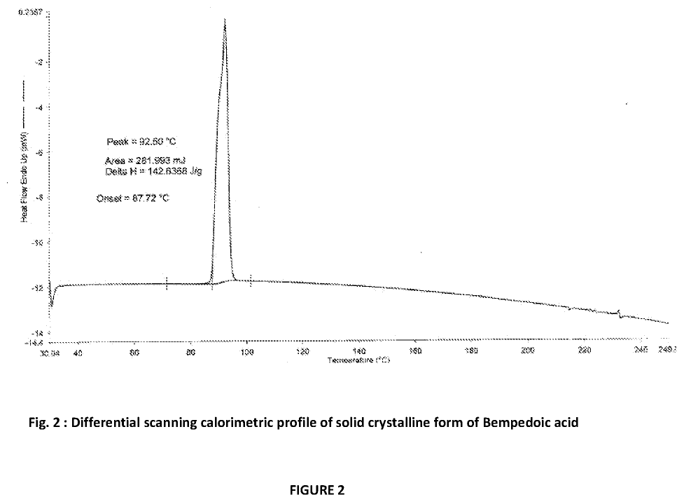

Yet another aspect of the present invention crystalline form of Bempedoic acid characterized by Differential scanning calorimetry (DSC) thermogram as depicted in FIG. 2.

Another aspect of the present provides a process for the preparation of crystalline form of Bempedoic acid comprising the steps of.
a) dissolving Bempedoic acid in a solvent,
b) optionally, adding second solvent,
c) heating the reaction mass,
d) cooling the reaction mass, and
e) isolating crystalline form of Bempedoic acid.

According to the present invention, solvent or second solvent is selected from alcohol such as methanol, ethanol, isopropanol, n-propanol, tertiary-butyl alcohol; ketone solvents such as acetone, methyl isobutyl ketone, ethyl methyl ketone; chlorinated solvents such as dichloromethane, chloroform, carbon tetrachloride; esters such as methyl acetate, ethyl acetate, n-propyl acetate, n-butyl acetate, t-butyl acetate; ether solvents such as tetrahydrofuran, 2-methyl tetrahydrofuran, dimethyl ether, diethyl ether, diisopropyl ether, methyl tert-butyl ether; nitriles such as acetonitrile, butyronitrile, isobutyronitrile, polar aprotic solvents such as dimethyl acetamide, dimethylsulfoxide, dimethylformamide, N-methyl-2-pyrrolidone, water or a mixture thereof.

Yet another aspect of the present invention provides novel process for the preparation of Bempedoic acid of formula I.

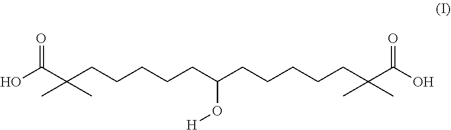 [SMILES: CC(C)(CCCCCC(O)CCCCCC(C)(C)C(=O)O)C(=O)O]

Scheme-1 is an illustration of the process for the preparation of Bempedoic acid according to another aspect of present invention.

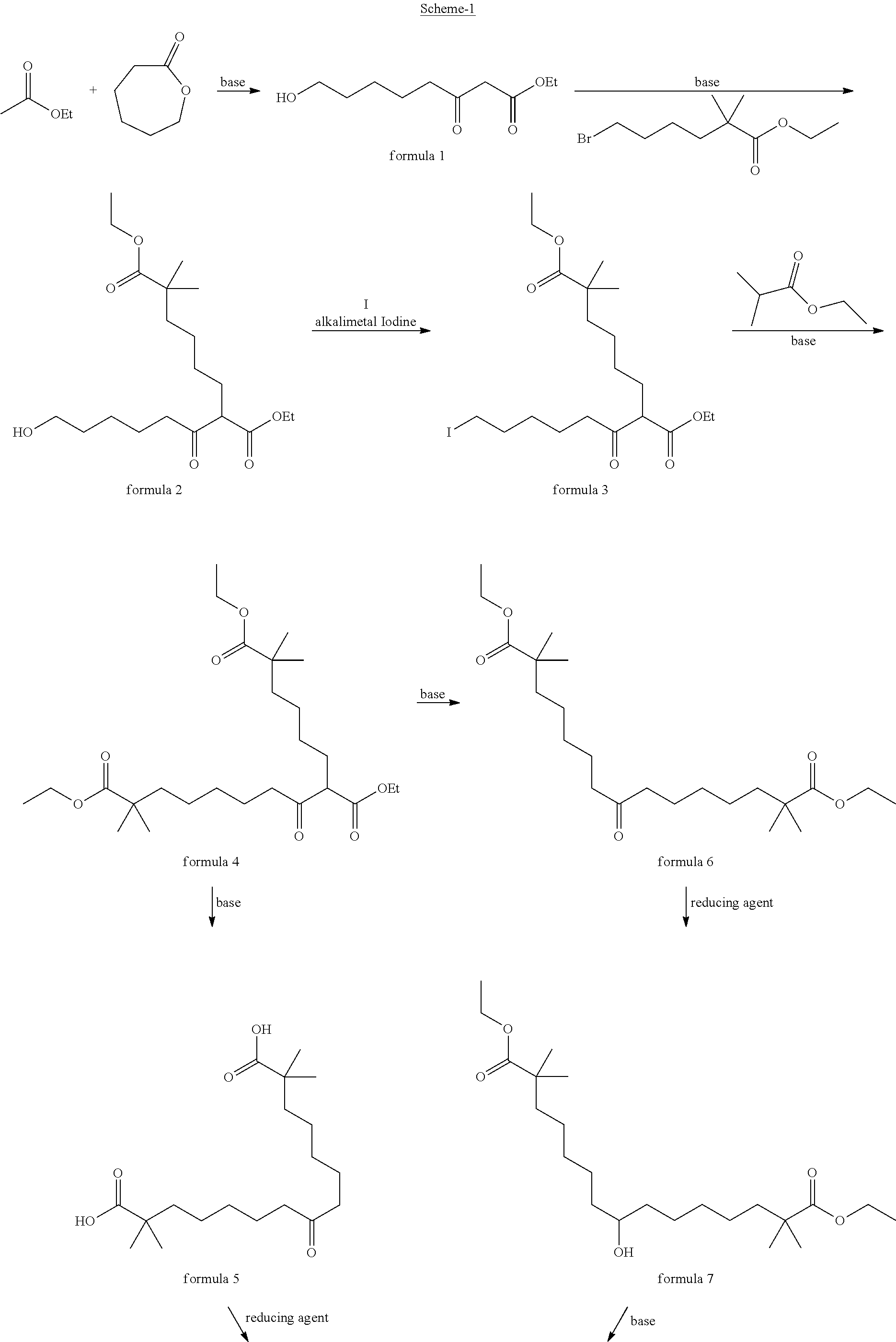

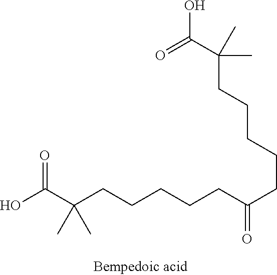 [SMILES: CC(C)(CCCCCC(=O)CCCCCC(C)(C)C(=O)O)C(=O)O]

Another aspect of the present invention provides a novel process for the preparation of Bempedoic acid (compound of formula I) comprising the steps of:
a) treating caprolactone with ethyl acetate in presence of base to give ethyl 8-hydroxy-3-oxooctanoate (formula 1),

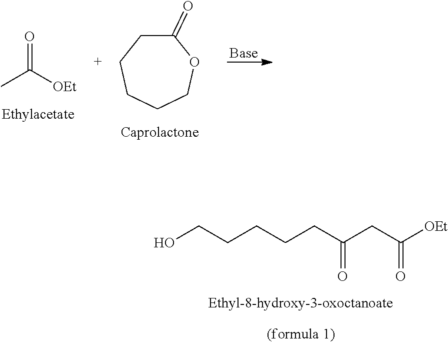

b) treating ethyl-8-hydroxy-3-oxooctanoate (formula 1) with ethyl 6-bromo-2,2-dimethylhexanoate to give diethyl 7-(6-hydroxyhexanoyl)-2,2-dimethyloctanedioate (formula 2),

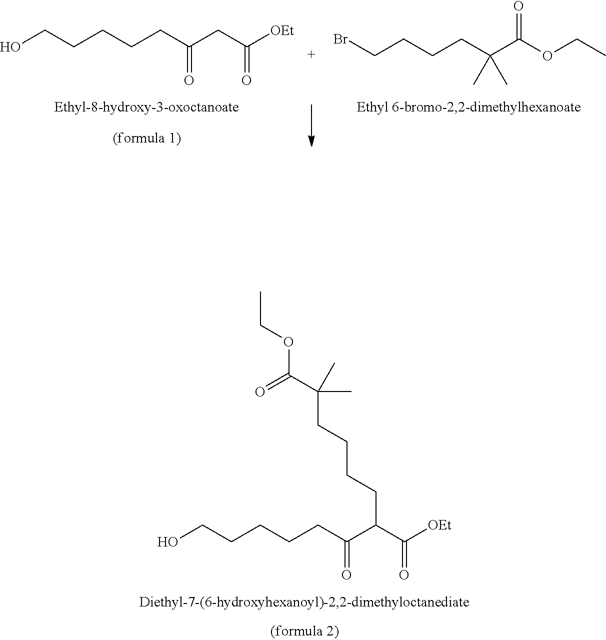

c) reacting diethyl 7-(6-hydroxyhexanoyl)-2,2-dimethyloctanedioate (formula 2) with alkali metal halide or tetrabutylammonium halide salt to give diethyl 7-(6-hydroxyhexanoyl)-2,2-dimethyloctanedioate (formula 3),

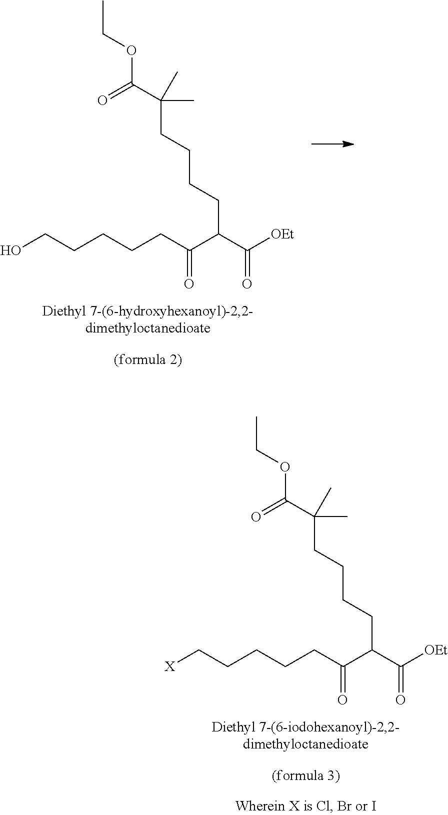

d) treating diethyl 7-(6-hydroxyhexanoyl)-2,2-dimethyloctanedioate (formula 3) with ethyl isobutyrate in presence of a base to give triethyl 2,14-dimethyl-8-oxopentadecane-2,7,14-tricarboxylate (formula 4),

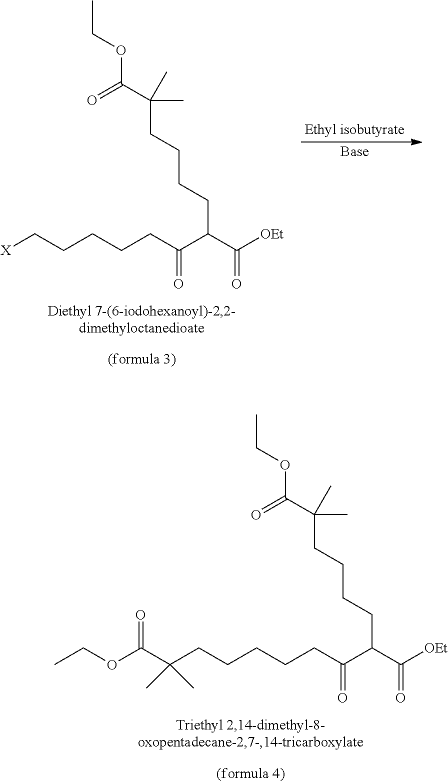

e) treating triethyl 2,14-dimethyl-8-oxopentadecane-2,7,14-tricarboxylate (formula 4) with abase to give 2,14-dimethyl-8-oxopentadecanedioic acid (formula 5),

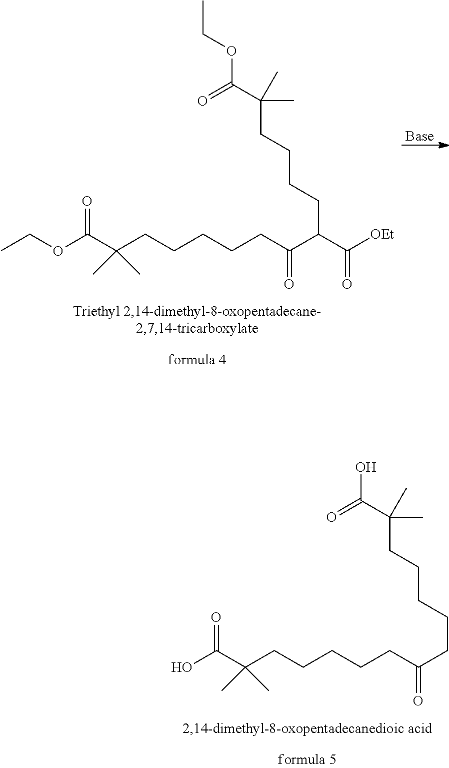 [SMILES: CCOC(=O)C(CCCCC(C)(C)C(=O)OCC)C(=O)CCCCCC(C)(C)C(=O)OCC ; "Base" ; CC(C)(CCCCCC(=O)CCCCCC(C)(C)C(=O)O)C(=O)O]

f) optionally treating, triethyl 2,14-dimethyl-8-oxopentadecane-2,7,14-tricarboxylate (formula 4) with a base to give diethyl 2,2,14,14-tetramethyl-8-oxopentadecanedioate (formula 6),

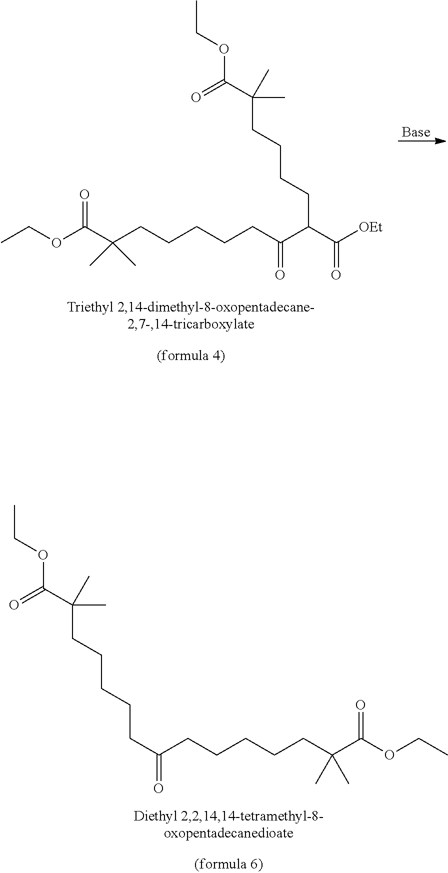 [SMILES: CCOC(=O)C(CCCCC(C)(C)C(=O)OCC)C(=O)CCCCCC(C)(C)C(=O)OCC ; "Base" ; CCOC(=O)C(C)(C)CCCCCC(=O)CCCCCC(C)(C)C(=O)OCC]

g) treating diethyl 2,2,14,14-tetramethyl-8-oxopentadecanedioate (formula 6) with a reducing reagent to give diethyl 8-hydroxy-2,2,14,14-tetramethylpentadecanedioate (formula 7),

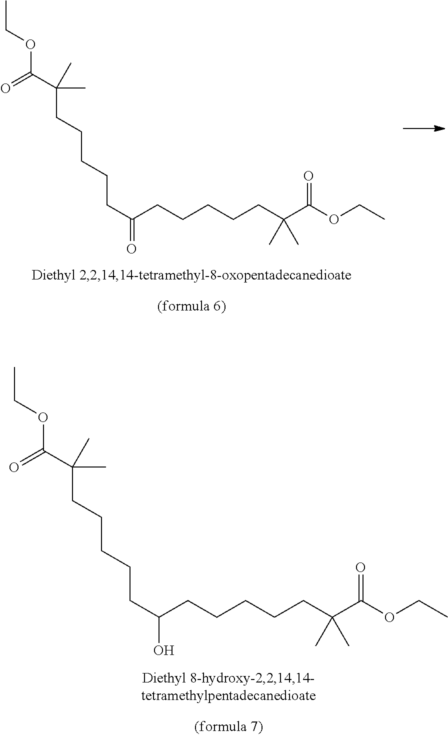

h) optionally treating, 2,14-dimethhyl-8-oxopentadecanedioic acid (formula 5) with a reducing reagent to give Bempedoic acid (compound of formula I), and
i) treating diethyl 8-hydroxy-2,2,14,14-tetramethylpentadecanedioate (formula 7) with a base to give (compound of formula I).

Another aspect of the present invention provides a process for the preparation of crystalline form of Bempedoic acid by using 7-iodo-2,2-dimethylheptanoic acid ethyl ester compound of formula (2a).

Scheme-2 is an illustration of the process for the preparation of crystalline form of Bempedoic acid according to another aspect of present invention.

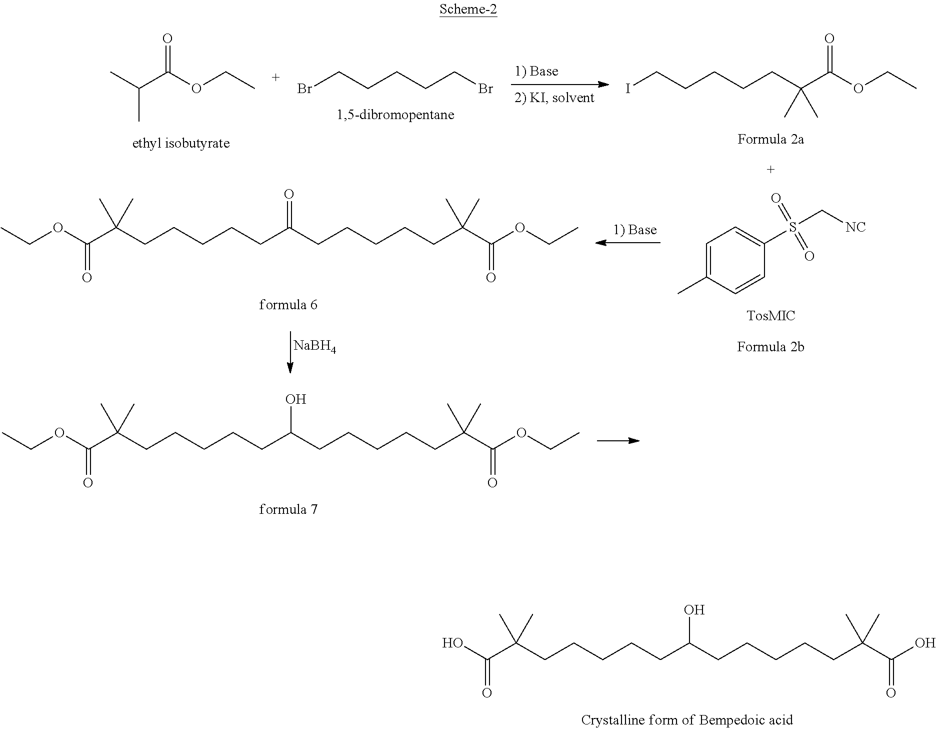

Another aspect of the present invention provides a novel process for the preparation of crystalline form of Bempedoic acid comprising the steps of:
a) treating ethyl isobutyrate with 1,5-dibromopentane in presence of base to give compound of formula 2a,

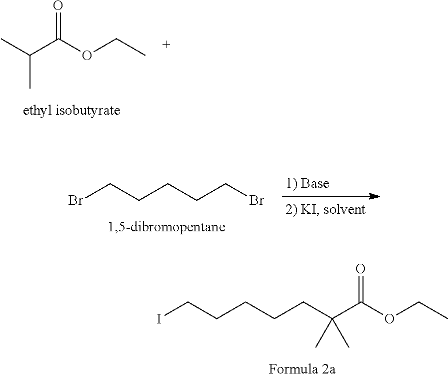

b) treating compound of formula 2a with compound of formula 2b in presence of a base to give compound of formula 6,

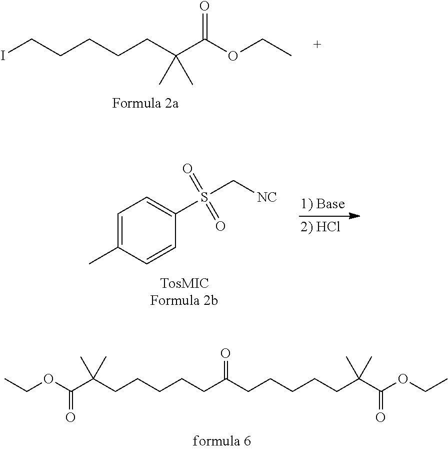

c) treating compound of formula 6 with a base to give compound of formula 7,

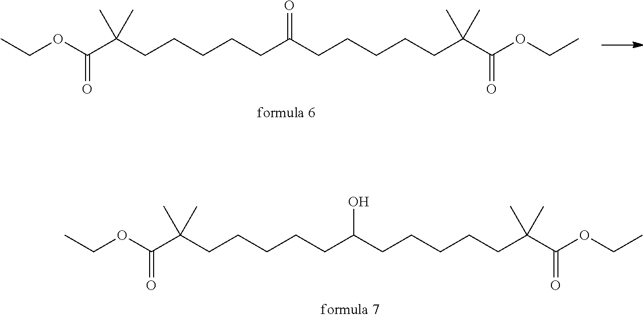

d) converting compound of formula 7 to crystalline form of Bempedoic acid

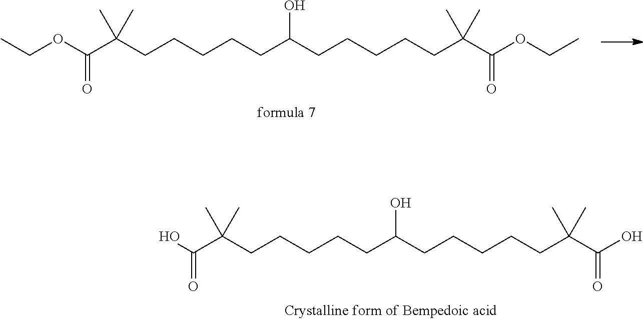 [SMILES: CCOC(=O)C(C)(C)CCCCCC(O)CCCCCC(C)(C)C(=O)OCC ; CC(C)(CCCCCC(O)CCCCCC(C)(C)C(=O)O)C(=O)O]

According to the present invention, solvent or organic solvent is selected from alcohol such as methanol, ethanol, isopropanol, n-propanol, tertiary-butyl alcohol; ketone solvents such as acetone, methyl isobutyl ketone, ethyl methyl ketone; chlorinated solvents such as dichloromethane, chloroform, carbon tetrachloride; esters such as methyl acetate, ethyl acetate, n-propyl acetate, n-butyl acetate, t-butyl acetate; ether solvents such as tetrahydrofuran, 2-methyl tetrahydrofuran, dimethyl ether, diethyl ether, diisopropyl ether, methyl tert-butyl ether; nitriles such as acetonitrile, butyronitrile, isobutyronitrile, polar aprotic solvents such as dimethyl acetamide, dimethylsulfoxide, dimethylformamide, N-methyl-2-pyrrolidone, water or a mixture thereof.

According to the present invention, base is selected from alkali metal hydrides, alkali metal alkoxides, alkali metal hydroxides, alkali metal oxides, alkali metal carbonates, quaternary ammonium alkoxides, quaternary ammonium hydroxides, quaternary phosphonium alkoxides, quaternary phosphonium hydroxides, tertiary amines or mixtures thereof. Preferred bases include sodium hydride, potassium hydride, sodium butoxide, potassium butoxide, sodium methoxide, potassium methoxide, sodium ethoxide, potassium ethoxide, sodium propoxide, potassium propoxide, sodium beta-hydroxyethoxide, potassium beta-hydroxyethoxide, sodium hydroxide, potassium hydroxide, sodium oxide, potassium oxide, sodium carbonate, potassium carbonate, benzyl trimethylammonium methoxide, benzyl trimethylammonium hydroxide, methyl triphenylphosphonium methoxide, triphenylphosphonium hydroxide, triethylamine, N-methyl-di-isopropylamine, tri-n-butylamine, tri-n-octylamine, 1,4-diazabicyclo(2.2.2)octane (DABCO), 1,5-diazabicyclo(4.3.0)non-5-ene(DBN), 1,8-diazabicyclo(5.4.0)undec-7-ene(DBU), N-methylpyrrolidine, N-methylpiperidine, N-methylmorpholine, N,N-dimethylpiperazine, pentamethyl guanidine, 2,6-lutidine, 2,4,6-collidine or mixtures thereof.

According to the present invention, alkali metal halide is selected from sodium iodide, potassium iodide, Tetrabutylammonium halide selected from Tetrabutylammonium iodide, Tetrabutylammonium bromide, or mixtures thereof.

According to the present invention, reducing reagent is selected from triacetoxy sodium boron hydride, triacetoxy tetramethylammonium borohydride, sodium cyanoborohydride, sodium borohydride, lithium borohydride, trimethoxy sodium boron hydride, tris ethyl lithium borohydride, borohydride reagents, lithium aluminum hydride, diisopropyl aluminum hydride, bis (2-methoxyethoxy) aluminum hydride, sodium aluminum hydride reagent, using a metal catalyst and a hydrogen source in the catalytic reduction or mixtures thereof.

According to the present invention, compound of formula XE wherein P is selected from the group consisting of alkyl, substituted alkyl, C₁-C₁₂ aryl, substituted C₁-C₁₂ aryl. As used herein, the term “alkyl” and its derivatives and derivatives in all carbon chains means a straight or branched saturated or unsaturated hydrocarbon chain, not otherwise defined. As long as the carbon chain contains 1 to 12 carbon atoms. Examples of alkyl substituents used herein include —CH₃, —CH₂—CH₃, —CH₂—CH₂—CH₃, —CH(CH₃)₂, —C(CH₃)₃, —(CH₂)₃—CH₃, —CH₂—CH(CH₃)₂, —CH(CH₃)—CH₂—CH₃, —CH═CH₂, and —C≡C—CH₃ Can be mentioned. The term “aryl” as used herein, unless otherwise defined, contains 1 to 14 carbon atoms and may contain 1 to 5 heteroatoms (provided that When the number is 1, the aromatic ring contains at least 4 heteroatoms, and when the number of carbon atoms is 2, the aromatic ring contains at least 3 heteroatoms and the number of carbons is 3 The aromatic ring contains at least 2 heteroatoms, and when the number of carbon atoms is 4, the aromatic ring contains at least 1 heteroatom).

The term “C₁-C₁₂ aryl” as used herein, unless otherwise defined, includes phenyl, benzyl, naphthalene, 3,4-methylenedioxyphenyl, pyridine, biphenyl, quinoline, pyrimidine, quinazoline, thiophene, furan, Pyrrole, pyrazole, imidazole and tetrazole.

Yet another aspect of the present invention provides novel process for the preparation of diethyl 2,2,14,14-tetramethyl-8-oxopentadecanedioate by using any one of the intermediate selected from compound of formula XA, compound of formula XB, compound of formula XC, and compound of formula XE.

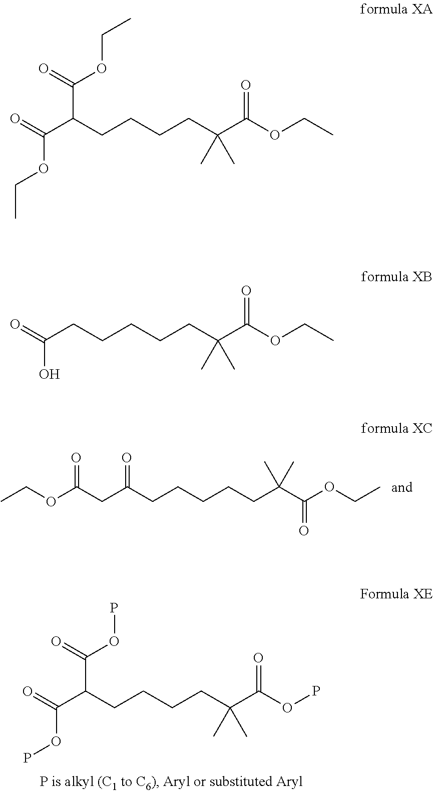

Scheme-3 is an illustration of the process for the preparation of diethyl 2,2,14,14-tetramethyl-8-oxopentadecanedioate according to another aspect of present invention.

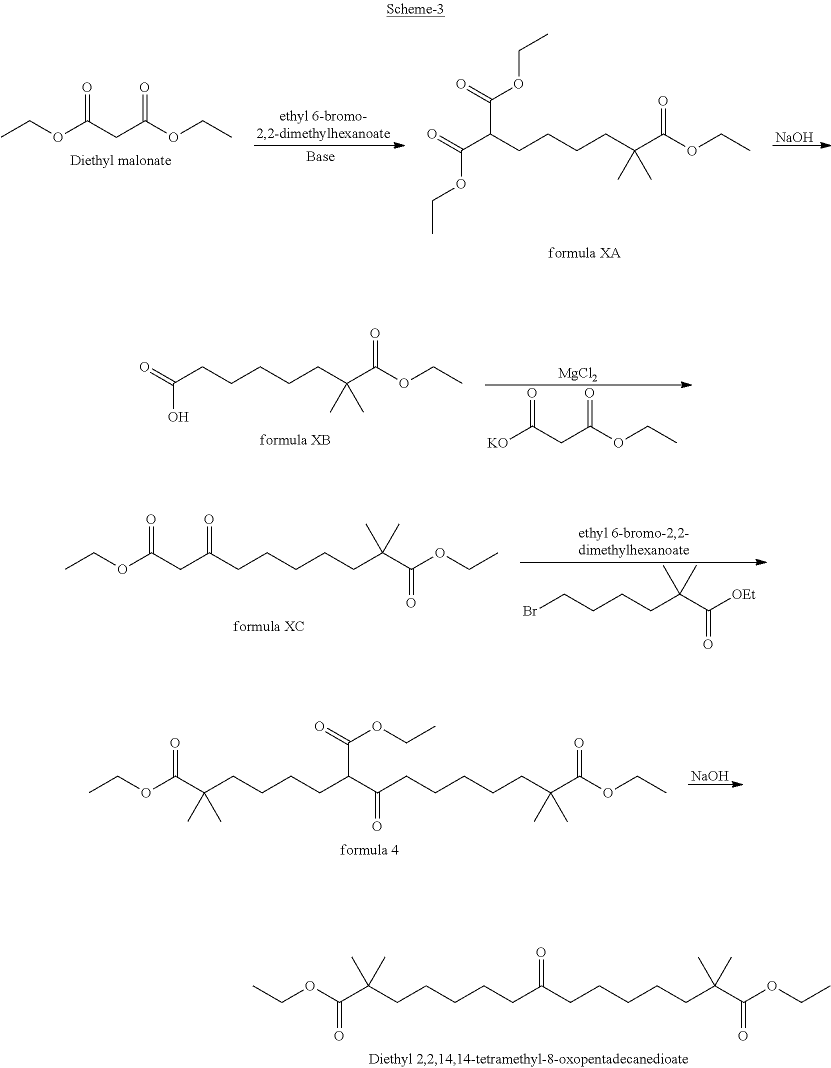

Yet another aspect of the present invention provides a novel process for the preparation of diethyl 2,2,14,14-tetramethyl-8-oxopentadecanedioate comprising the steps of:
a) treating diethyl malonate with 7-bromo-3-methyl heptan-2-one in presence of base to give compound of formula XA,

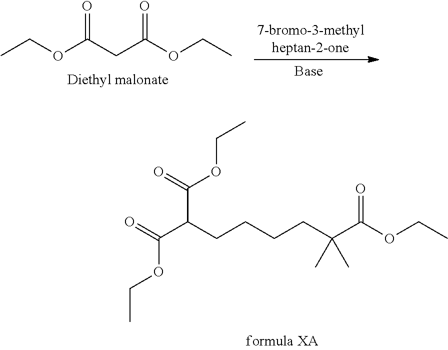 [SMILES: CCOC(=O)CC(=O)OCC ; CCOC(=O)C(CCCCC(C)(C)C(=O)OCC)C(=O)OCC]

b) treating compound of formula XA with a base to give compound of formula XB,

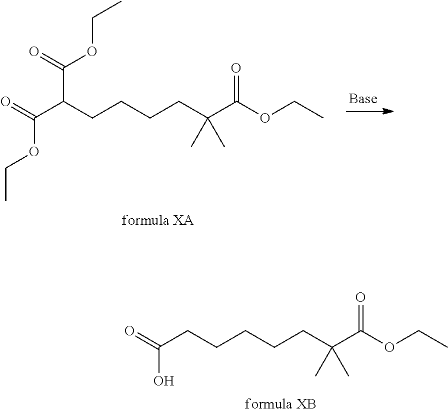 [SMILES: CCOC(=O)C(CCCCC(C)(C)C(=O)OCC)C(=O)OCC ; CCOC(=O)C(C)(C)CCCCCC(=O)O]

c) reacting compound of formula XB with potassium 3-ethoxy-3-oxopropanoate to give compound of formula XC

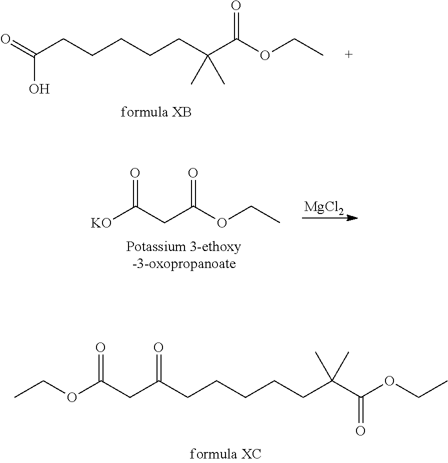

d) treating compound of formula XC with 7-bromo-3-methyl heptan-2-one in presence of a base to give compound of formula 4,

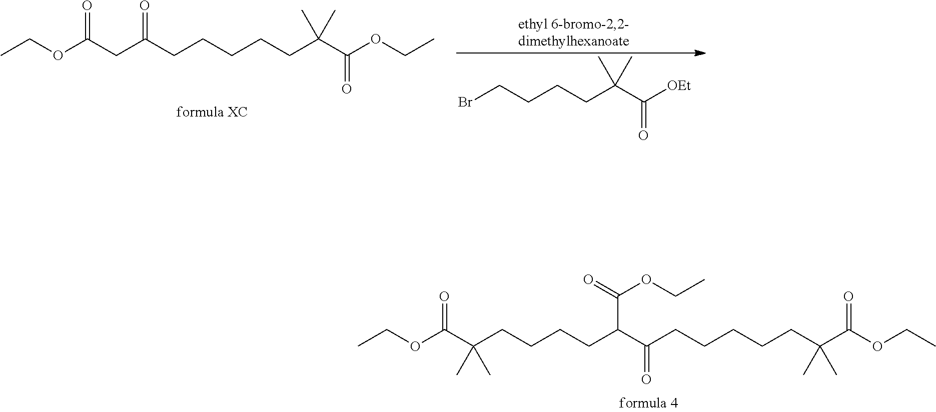 [SMILES: CCOC(=O)CC(=O)CCCCCC(C)(C)C(=O)OCC ; CCOC(=O)C(C)(C)CCCCBr ; CCOC(=O)C(CCCCC(C)(C)C(=O)OCC)C(=O)CCCCCC(C)(C)C(=O)OCC]

e) treating compound of formula 4 with a base to give diethyl 2,2,14,14-tetramethyl-8-oxopentadecanedioate, and
f) using diethyl 2,2,14,14-tetramethyl-8-oxopentadecanedioate intermediate to prepare Bempedoic acid.

Yet another aspect of the present invention provides a novel compound of formula XE and process for the preparation thereof

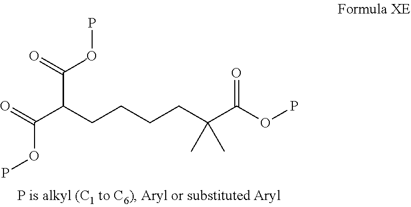

According to the present invention, compound of formula XE is uses as an intermediate to prepare Bempedoic acid or pure Bempedoic acid or crystalline form of Bempedoic acid.

Another aspect of the present invention provides novel process for the preparation of compound of formula XE by using novel intermediates of present invention or any prior art process.

Yet another aspect of the present invention provides novel process for the preparation of 2,14-dimethyl-8-oxopentadecanedioic acid (compound of formula 5).

Another aspect of the present invention provides novel process for the preparation of diethyl 2,2,14,14-tetramethyl-8-oxopentadecanedioate (compound of formula 6).

Scheme-4 is an illustration of the process for the preparation of 2,14-dimethyl-8-oxopentadecanedioic acid (compound of formula 5) or diethyl 2,2,14,14-tetramethyl-8-oxopentadecanedioate (compound of formula 6) according to another aspect of present invention.

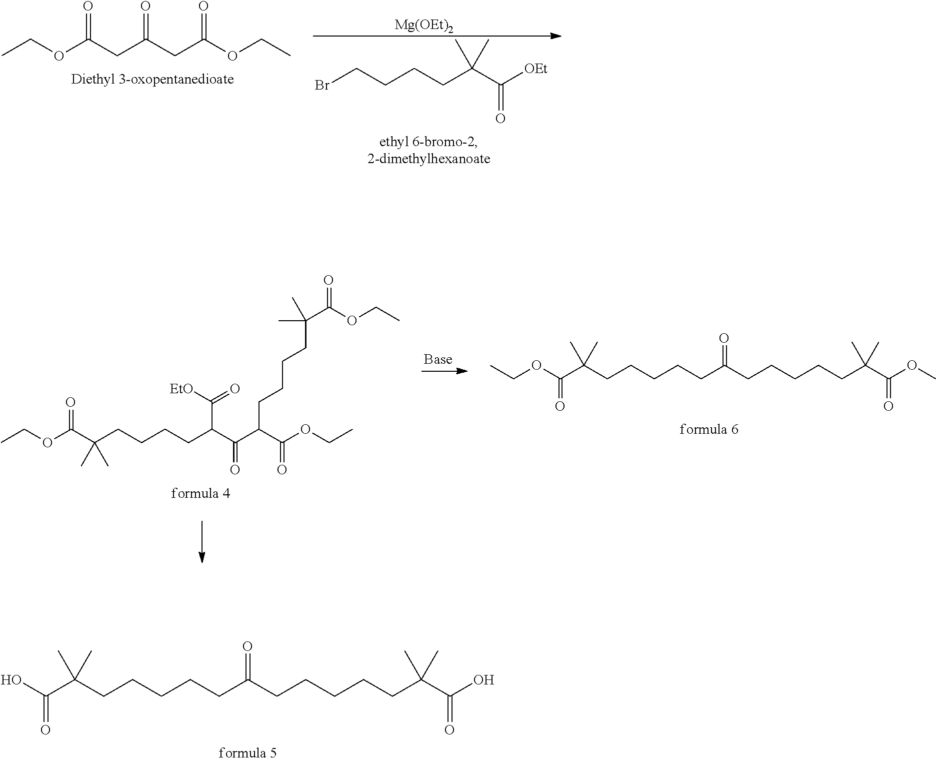

Yet another aspect of the present invention provides a novel process for the preparation of 2,14-dimethyl-8-oxopentadecanedioic acid (compound of formula 5) or diethyl 2,2,14,14-tetramethyl-8-oxopentadecanedioate (compound of formula 6) comprising the steps of:
a) treating diethyl 3-oxopentanedioate with ethyl 6-bromo-2,2-dimethylhexanoate to give compound of formula 4, and
b) treating compound of formula 4 with a base to give compound of formula 5 or compound of formula 6.

According to the process of the present invention, Bempedoic acid is having high purity.

Another aspect of the present invention crystalline form Bempedoic acid is prepared from Bempedoic acid or pure Bempedoic acid as prepared in present invention or from any other prior-art process.

### Experimental Method

1) HPLC Instrument and Method Details:

Instrument: HPLC equipped with Pump, injector, UV detector and Recorder.

Column: Zorbax SB-Aq (4.6×250 mm), 5 μm.

Wavelength: UV Detector 215 nm

Flow rate: 1.5 mL/min

Injection volume: 5 μL.

Auto sampler temperature: 10° C.

Column oven temperature: 20° C.

The NMR spectrum was recorded by using a Bruker Avance III HD 500 MHz instrument.

Having thus described the various aspects of the present invention, the following examples are provided to illustrate specific embodiments of the present invention. They are not intended to be limiting in any way.

### EXAMPLES

### Example-1: Preparation of Sodium Salt of Bempedoic Acid

Bempedoic acid (1.0 g, 0.0029 mol), MeOH (10 mL), sodium hydroxide solution (0.11 g, 0.0028 mol) and water (1 ml) were added, and the reaction mixture was stirred for 30 min at ambient temperature and then concentrated the reaction mass under reduced pressure. The obtained solid was dried to give sodium salt of Bempedoic acid.

¹H-NMR (500 MHz, DMSO-d6): δ 3.33 (s, 1H), 1.37 (m, 20H), 1.04 (m, 12H).

### Example-2: Preparation of Potassium Salt of Bempedoic Acid

Bempedoic acid (1.0 g, 0.0029 mol) in MeOH (10 mL), KOH solution (0.16 g, 0.0028 mol) and water (1 ml) was added. The reaction mixture was stir for 1 hr at ambient temperature and then concentrated the reaction mass under reduced pressure. The obtained solid was dried to give potassium salt of Bempedoic acid.

¹H-NMR (500 MHz, DMSO-d6): δ 3.34 (s, 1H), 1.37 (m, 20H), 1.04 (m, 12H).

### Example-3: Preparation of Calcium Salt of Bempedoic Acid

Bempedoic acid (1.0 g, 0.0029 mol) was dissolved in MeOH (10 mL), added NaOH solution (0.11 g, 0.0029 mol) and water (2 ml) was added to the reaction mass. Reaction mixture stirred for 15 min at 50° C. Slowly added calcium acetate (0.22 g, 0.0014 mol) and water to reaction mixture and stirring continued for 30 min at 50° C. Reaction mixture was concentrated under reduced pressure, stripped out with acetone and then degassed to give calcium salt of Bempedoic acid.

¹H-NMR (500 MHz, DMSO-d6): δ 3.42 (s, 1H), 1.35 (m, 20H), 1.00 (m, 12H).

### Example-4: Preparation of Piperazine Salt of Bempedoic Acid

Bempedoic acid (1.0 g, 0.0029 mol) was suspended in THF (40 mL), added piperazine solution (0.24 g, 0.0029 mol) and THF (5 mL) to reaction mass at ambient temperature. Reaction mass was stirred for 5 h and then filtered. The obtained solid was dried to give piperazine salt of Bempedoic acid.

1H-NMR (500 MHz, MeOD): δ 3.51 (m, 1H), 3.06 (s, 8H), 1.52 (m, 4H), 1.46 (m, 16H), 1.38 (m, 12H).

### Example-5: Preparation of Bis-Piperazine Salt of Bempedoic Acid

Bempedoic acid (1.0 g, 0.0029 mol) was dissolved in MeOH (10 mL), added piperazine (0.49 g, 0.0058 mol) to the reaction mass at ambient temperature. Reaction mixture was stirred for 1 h at 50° C. Reaction mass was concentrated under reduced pressure to give bis-piperazine salt of Bempedoic acid.

¹H-NMR (500 MHz, DMSO-d⁶): δ 3.33 (m, 1H), 2.67 (s, 16H), 1.40 (m, 20H), 1.04 (m, 12H).

### Example-6: Preparation of Bis-Tert-Butyl Amine Salt of Bempedoic Acid

Bempedoic acid (1.0 g, 0.0029 mol) was dissolved in MeOH (10 mL), added t-butyl amine (0.42 g, 0.0058 mol) to reaction mass. Reaction mixture was stir for 5 h at ambient temperature. Then reaction mass was concentrated under reduced pressure to give bis-tert-butylamine salt of Bempedoic acid.

¹H-NMR (500 MHz, MeOD): δ 3.51 (m, 1H), 1.50 (m, 9H), 1.45 (m, 29H), 1.31 (m, 12H)

### Example-7: Process for the Preparation of Bempedoic Acid

Example 7a: Process for the preparation of ethyl 8-hydroxy-3-oxooctanoate Ethyl acetate (30.0 g, 0.34 mol) and THF (300 mL) was added to LDA at −65° C. Stirred the reaction mass for 1 h and then added caprolactone at −65° C. Reaction mass was continued for 1 h and then quenched with ammonium chloride solution (50 mL). Allowed to come ambient temperature, diluted with water (200 mL) and extracted with EtOAc (200 mL). The organic layer was concentrated to give gummy mass of ethyl 8-hydroxy-3-oxooctanoate (67.3 g).

¹H-NMR (500 MHz, DMSO-d6) δ 4.20 (q, 2H), 4.07 (m, 1H), 3.66 (m, 2H), 3.44 (s, 2H), 2.58 (m, 2H), 2.3 (m, 1H), 1.60 (m, 6H), 1.39 (m, 3H), MS: 203.2 [M+H]+.

### Example 7b: Process for the Preparation of diethyl 7-(6-hydroxyhexanoyl)-2,2-dimethyloctanedioate

Ethyl 8-hydroxy-3-oxooctanoate (35.0 g, 0.173 mol) was dissolved in DMF (350 mL), ethyl 6-bromo-2,2-dimethylhexanoate (47.8 g, 0.190 mol) and K2CO3 (35.8 g, 0.259 mol) was added. Reaction mixture was stirred at 60° C. for 16 h, after completion of reaction, organic layer was separated and washed with water, brine and then concentrated to give diethyl 7-(6-hydroxyhexanoyl)-2,2-dimethyloctanedioate (28.4 g).

¹H-NMR (500 MHz, DMSO-d6) δ 4.18 (q, 2H), 4.10 (m, 2H), 3.66 (m, 2H), 3.40 (m, 1H), 2.56 (m, 2H), 1.83 (m, 2H), 1.63-1.48 (m, 6H), 1.26 (m, 2H), 1.14 (m, 10H), 1.15 (s, 6H); MS: 371.3 [M−H]+.

### Example 7c: Preparation of diethyl 7-(6-iodohexanoyl)-2,2-dimethyloctanedioate

Diethyl 7-(6-hydroxyhexanoyl)-2,2-dimethyloctanedioate (25.0 g, 0.067 mol) was dissolved in mixture of DCM (250 mL), DIPEA (10.3 g, 0.081 mol). Reaction mass was cooled to 0° C. and added methane sulphonyl chloride (8.4 g, 0.073 mol). Reaction mass was stir for 1 h and then quenched with 1 N HCl (125 mL). Organic layer was separated and concentrated to give residue. Obtained residue was dissolve in acetone (620 mL) and added potassium iodide (22.8 g, 0.137 mol). Reaction mixture was refluxed for 24 hr and concentrated to give residue. The residue was dissolve in EtOAc (310 mL) and washed with 20% sodium thiosulfate solution then by water (150 mL). Organic layer was concentrated to give diethyl 7-(6-iodohexanoyl)-2,2-dimethyloctanedioate (29.1 g) as oil.

¹H-NMR (500 MHz, CDCl₃): δ 4.20 (q, 2H), 4.12 (q, 4H), 3.42 (m, 1H), 3.21 (m, 2H), 2.51 (m, 2H), 1.84 (m, 2H), 1.60 m, 4H), 1.38 (m, 2H), 1.29 (m, 2H), 1.26 (m, 10H), 1.12 (s, 12H); MS: 500.1 [M+NH4]⁺.

### Example 7d: Preparation of triethyl 2,14-dimethyl-8-oxopentadecane-2,7,14-tricarboxylate

Diethyl 7-(6-iodohexanoyl)-2,2-dimethyloctanedioate (1.0 g, 0.002 mol) and ethyl isobutyrate (0.36 g, 0.0031 mol) was dissolved in THF (10 mL), cool the reaction mass to −60° C. and added LDA (2.6 mL, 0.0051 mol), stir the reaction mas for 16 hrs after quenched with ammonium chloride solution (20 mL) and extracted with EtOAc (2×20 mL). Organic layer was washed with brine and then concentrated to give triethyl 2,14-dimethyl-8-oxopentadecane-2,7,14-tricarboxylate (0.81 g) as oil.

¹H-NMR (500 MHz, CDCl₃): δ 4.21 (q, 2H), 4.11 (q, 4H), 3.40 (m, 1H), 2.51 (m, 2H), 1.84 (m, 2H), 1.60 (m, 8H), 1.24 (m, 16H), 1.12 (s, 12H); MS: 469.4 [M−H]⁺.

### Example 7e: Preparation of 2,2,14,14-tetramethyl-8-oxopentadecanedioic acid

Triethyl 2,14-dimethyl-8-oxopentadecane-2,7,14-tricarboxylate (0.5 g, 0.0011 mol) was dissolved in ethanol (8 mL). Added KOH (0.59 g, 0.106 mol) and water (2 mL) to reaction mass. Reaction mass was refluxed for 16 h, cooled to ambient temperature and water was added (10 mL). Adjust the reaction mass pH to 2-3 and then extracted with DCM, organic layer was concentrated to give 2,2,14,14-tetramethyl-8-oxopentadecanedioic acid (0.31 g).

¹H NMR (500 MHz, CDCl₃): δ 2.38 (m, 4H), 1.45 (m, 8H), 1.34 (m, 8H), 1.12 (s, 12H); MS: 460.2 [M+NH4]⁺.

### Example 7f: Preparation of diethyl 8-hydroxy-2,2,14,14-tetramethylpentadecanedioate

Diethyl 2,2,14,14-tetramethyl-8-oxopentadecanedioate (9.0 g, 0.02 mol) was dissolved in methanol (100 mL), cool the reaction mass to 0° C. and added NaBH₄ (0.83 g, 0.02 mol). Extracted the reaction mass with DCM. Combined organic layer, and stripped off solvent under reduced pressure to give diethyl 8-hydroxy-2,2,14,14-tetramethylpentadecanedioate (8.1 g) as an oily mass.

¹H NMR (500 MHz, CDCl₃): δ 4.13 (m, 4H), 3.59 (m, 1H), 1.59-1.42 (m, 8H), 1.25 (m, 16H), 1.16 (s, 12H); MS: 418.3 [M+NH4]⁺.

### Example 7g: Preparation of Bempedoic Acid: Method A

2,2,14,14-tetramethyl-8-oxopentadecanedioic acid (1.1 g, 0.0032 mol) was dissolved in methanol. Cool the reaction mass to 0° C. and added NaBH₄ (0.46 g, 0.0122 mol). Stir the mass for 5 h at ambient temperature and then added 1 N HCl (100 mL). Organic layer was concentrated under reduced pressure to give Bempedoic acid (8.1 g) as an oily mass.

¹H-NMR (500 MHz, DMSO-d6): δ 4.13 (m, 1H), 1.43 (m, 4H), 1.25 (m, 16H), 1.06 (s, 12H); MS: 343.2 [M−1]⁻.

### Example 7h: Preparation of Bempedoic Acid: Method B

Diethyl 8-hydroxy-2,2,14,14-tetramethylpentadecanedioate (8.0 g, 0.019 mol) was dissolved in EtOH (240 mL), add KOH (10.0 g, 0.17 mol) and water (8 mL) to reaction mixture, mixture was refluxed for 16 h. Reaction mass was concentrated under reduced pressure. Residue obtained was diluted with water (80 mL) and acidified with 1N HCl and extracted with DCM and organic layer was removed under reduced pressure to give residue which was crystallized with DIPE (160 mL) to give 8-hydroxy-2,2,14,14-tetramethylpentadecanedioic acid (4.8 g) as a white solid.

¹H-NMR (500 MHz, DMSO-d6): δ 4.13 (m, 1H), 1.43 (m, 4H), 1.25 (m, 16H), 1.06 (s, 12H); MS: 343.2 [M−1]⁻.

### Example 8: Process for the Preparation of triethyl 2,14-dimethyl-8-oxopentadecane-2,7,14-tricarboxylate

### Example 8a: Process for the Preparation of tetraethyl 2,14-dimethyl-8-oxopentadecane-2,7,9,14-tetracarboxylate

Diethylmalonate (4.5 g, 0.028 mol) was dissolved in DMF (45 mL). Added ethyl 6-bromo-2,2-dimethylhexanoate (7.77 g, 0.030 mol) and K2CO3 (5.82 g, 0.042 mol). Reaction mixture was stirred for 16 h at 60° C., separate both layers and organic layer was concentrated to give triethyl 6-methylheptane-1,1,6-tricarboxylate (9.1 g) as an oil.

¹H NMR (500 MHz, CDCl₃): δ 4.23-4.10 (m, 6H), 3.31 (m, 1H), 1.92 (m, 2H), 1.53 (m, 2H), 1.29 (m, 13H), 1.18 (s, 6H); MS: 348 [M+NH₄]⁺.

### Example 8b: Preparation of 8-ethoxy-7,7-dimethyl-8-oxooctanoic acid

Triethyl 6-methylheptane-1,1,6-tricarboxylate (9.0 g, 0.027 mol) was dissolved in ethanol (45 mL), added NaOH (2.72 g, 0.068 mol) and water (27 mL). Reaction mixture was stir for 18 h at ambient temperature. Reaction mass was acidified with 1N HCl and extracted with EtOAc. Organic layer was concentrated to give 8-ethoxy-7,7-dimethyl-8-oxooctanoic acid (5.2 g).

¹H-NMR (500 MHz, CDCl₃): δ 4.11 (q, 2H), 2.34 (t, 2H), 1.63 (m, 2H), 1.52 (m, 2H), 1.36 (m, 2H), 1.27 (m, 5H), 1.20 (s, 6H); MS: 231.2 [M+1]⁺.

### Example 8c: Preparation of diethyl 2,2-dimethyl-8-oxodecanedioate

8-ethoxy-7,7-dimethyl-8-oxooctanoic acid (2.0 g, 0.0087 mol) was dissolved in THF (20 mL), CDI (1.55 g, 0.0095 mol) was added. Reaction mass was stirred for 2 h at ambient temperature. In another flask potassium malonate (2.96 g, 0.0174 mol) and MgCl2 (1.65 g, 0.0174 mol) was dissolved in THF (30 mL), slowly triethylamine (1.75 g, 0.0173 mol) was added and stirred for 2 h at ambient temperature. Reaction mixture was cooled and quenched with 1N HCl solution and then extracted with EtOAc. Organic layer was removed to give diethyl 2,2-dimethyl-8-oxodecanedioate.

¹H-NMR (500 MHz, CDCl₃): δ 4.20 (q, 2H), 4.11 (q, 2H), 3.43 (s, 2H), 2.53 (t, 2H), 1.64 (m, 2H), 1.52 (m, 2H), 1.25 (m, 9H), 1.16 (s, 6H); MS: 318.1 [M+NH₄]⁺.

### Example 8d: Preparation of triethyl 2,14-dimethyl-8-oxopentadecane-2,7,14-tricarboxylate

Diethyl 2,2-dimethyl-8-oxodecanedioate (1.0 g, 0.0033 mol) was dissolved in DMF (10 mL), K2CO3 (0.69 g, 0.005 mol), ethyl 6-bromo-2,2-dimethylhexanoate (0.88 g, 0.0035 mol) were added and stirred for 2 h at 60° C. Reaction mixture was cooled and diluted with water (60 mL) and extracted with EtOAc (2×20 mL). Organic layer was washed with water and then concentrated to give residue which was purified by chromatography to give triethyl 2,14-dimethyl-8-oxopentadecane-2,7,14-tricarboxylate (1.2 g).

¹H-NMR (500 MHz, CDCl₃): δ 4.21 (q, 2H), 4.11 (q, 4H), 3.40 (m, 1H), 2.51 (m, 2H), 1.84 (m, 2H), 1.60 (m, 8H), 1.24 (m, 16H), 1.12 (s, 12H); MS: 469.4 [M−H]⁺.

### Example 9: Process for the Preparation of diethyl 2,2,14,14-tetramethyl-8-oxopentadecanedioate

### Example 9a: Process for the Preparation of tetraethyl 2,14-dimethyl-8-oxopentadecane-2,7,9,14-tetracarboxylate

Diethyl 3-oxopentanedioate (4.0 g, 0.0198 mol) was dissolved in DMF (40 mL), Mg(OEt)2 (0.059 mol), ethyl 6-bromo-2,2-dimethylhexanoate (10.9 g, 0.043 mol) were added. Reaction mixture was stir for 20 h at 60° C. Reaction mixture was cooled and quenched with HCl solution. Reaction mass was extracted with EtOAc (80 mL), organic layer was separated and concentrated under reduced pressure. Residue obtained was purified with column chromatography to give tetraethyl 2,14-dimethyl-8-oxopentadecane-2,7,9,14-tetracarboxylate.

¹H NMR (500 MHz, CDCl₃): δ 4.20-4.08 (m, 8H), 3.68-3.46 (m, 2H), 1.85 (m, 4H), 1.24 (m, 18H), 1.17 (s, 12H); MS: 560 [M+NH4]⁺.

### Example 9b: Process for the Preparation of diethyl 2,2,14,14-tetramethyl-8-oxopentadecanedioate

Tetraethyl 2,14-dimethyl-8-oxopentadecane-2,7,9,14-tetracarboxylate (1.0 g, 0.0018 mol) was dissolved in ethanol (20 mL), added KOH (1.0 g, 0.018 mol) and water (5 mL). Reaction mixture was stirred for 16 h at 90-95° C. Reaction mass was cooled and acidified with HCl solution. It was extracted with DCM (50 mL) and then concentrated under reduced pressure to give diethyl 2,2,14,14-tetramethyl-8-oxopentadecanedioate (0.31 g).

¹H-NMR (500 MHz, CDCl₃): δ 4.13 (m, 4H), 2.38 (m, 4H), 1.64-1.49 (m, 10H), 1.25 (m, 14H), 1.16 (s, 12H); MS: 416.2 [M+NH4]⁺.

### Example 10: Process for the Preparation of Crystalline Bempedoic Acid

### Example-10a: Process for the Preparation of Preparation of ethyl 7-iodo-2,2-dimethylheptanoate (2a)

Ethyl isobutyrate (50.0 g, 0.43 mol) was dissolved in THF (500 mL), cooled the reaction mass to −40° C. and slowly added dissolved LDA (236.7 L, 0.473 mol). Stirred for 30 min and then 1,5-dibromopentane (108.8 g, 0.473 mol) was added. Reaction mixture was stirred at ambient temperature for overnight. Reaction mass was quenched with 20% NH₄Cl solution (250 mL) and extracted with EtOAc (2×250 mL). Organic layer was concentrated on rotavapour under reduced pressure.

The residue obtained was dissolved in acetone (400 mL) and added slowly KI (71.8 g). Reaction mass was stirred overnight at 50° C. and then cooled to ambient temperature. Organic solvent was removed on rotavapour and residue obtained was diluted with EtOAc (400 mL). Reaction mass was washed with water followed by brine and then concentrated to get ethyl 7-iodo-2,2-dimethylheptanoate (41.0 g) as oily mass. 1H NMR (500 MHz, CDCl3): δ 4.13 (q, 2H), 3.19 (t, 2H), 1.82 (m, 2H), 1.52 (m, 2H), 1.41 (m, 2H), 1.24 (m, 5H), 1.16 (s, 6H).

### Example-10b: Process for the Preparation of diethyl 2,2,14,14-tetramethyl-8-oxopentadecanedioate

Ethyl 7-iodo-2,2-dimethylheptanoate (31.2 g, 0.10 mol), TosMIC (27.0 g, 0.05 mol), and TBAI (3.76 g, 0.01 mol) was dissolved in THF (270 mL) LDA (0.12 mol). Reaction mixture was cool to 0° C., reaction mass was allowed to room temperature for 6 hrs and quenched the reaction mass with 20% NH4Cl (200 mL) followed by extracted with EtOAc (200 mL). Organic solvents was removed under reduced pressure to get residue that DCM (400 mL) was added. Added Conc. HCl (100 mL) to reaction mass under stirring at ambient temperature. Reaction mass was diluted with water (300 mL) and DCM layer was separated. Organic layer washed with NaHCO₃ (100 mL), water and then with brine. DCM was removed under reduced pressure to give residue. The residue was purified with column chromatography to give diethyl 2,2,14,14-tetramethyl-8-oxopentadecanedioate (14.5 g) as an oily mass. 1H NMR (500 MHz, CDCl3): δ 4.13 (m, 4H), 2.38 (m, 4H), 1.64-1.49 (m, 8H), 1.25 (m, 16H), 1.16 (s, 12H); MS: 416.2 [M+NH4]⁺.

### Example-10c: Process for the Preparation of diethyl 8-hydroxy-2,2,14,14-tetramethylpentadecanedioate

Diethyl 2,2,14,14-tetramethyl-8-oxopentadecanedioate (9.0 g, 0.02 mol) was dissolved in methanol (100 mL), reaction mass was cooled to 0° C. and added slowly NaBH₄ (0.83 g, 0.02 mol). Reaction mass was stirred for 3 h at ambient temperature and reaction mass was diluted with water (200 mL). Extracted the reaction mass with DCM (2×200 mL). Organic layer are stripped off under reduced pressure, thus obtained diethyl 8-hydroxy-2,2,14,14-tetramethylpentadecanedioate (8.1 g) as an oily mass. 1H NMR (500 MHz, CDCl3): δ 4.13 (m, 4H), 3.59 (m, 1H), 1.59-1.42 (m, 8H), 1.25 (m, 16H), 1.16 (s, 12H); MS: 418.3 [M+NH4]⁺.

### Example-10d: Process for the Preparation of Bempedoic Acid

Diethyl 8-hydroxy-2,2,14,14-tetramethylpentadecanedioate (8.0 g, 0.019 mol) was dissolved in EtOH (240 mL), reaction mass was stirred for 30 min and KOH (10.0 g, 0.17 mol), water (8 mL) was added and reaction mixture was refluxed for 16 h. Reaction mass was concentrated under reduced pressure. Water (80 mL) was added to the reaction mass. Reaction mass was acidified with 1N HCl, and extracted with DCM (2×100 mL). Organic layer was removed under reduced pressure to give residue which was crystallized with DIPE (160 mL) to give 8-hydroxy-2,2,14,14-tetramethylpentadecanedioic acid (4.8 g) as a white solid. 1H NMR (500 MHz, DMSO-d6): δ 12.02 (brs, 2H), 4.22-3.47 (m, 1H), 1.43 (m, 4H), 1.25 (m, 16H), 1.06 (s, 12H); MS: 343.2 [M−1].

### Example-10 e: Process for the Preparation of Crystalline Form of Bempedoic Acid

Bempedoic acid (1.0 g) was dissolved in diisopropyl ether (30 mL), reaction mass was stirred at 60° C. for 3 hr. Reaction mass was cooled to room temperature, Filtered the reaction mass and dried at 40° C., thus obtained crystalline Bempedoic acid (0.71 g).

### Example-10 f: Process for the Preparation of Crystalline Form of Bempedoic Acid

Bempedoic acid (1.0 g) was dissolved in acetone (12 mL), to the reaction mass water (12 ml) was added and heat the reaction mass to 50° C. Cooled the reaction mass to ambient temperature. Obtained solid was filtered and dried at 40° C. to give a white crystalline solid Bempedoic acid (0.6 g).

### Example-10 g: Process for the Preparation of Crystalline Form of Bempedoic Acid

Bempedoic acid (0.5 g) was dissolved in methanol (5 mL), stir the reaction mass and concentrate reaction mass on rotavapour at 40° C. The obtained solid was dried at 40° C. to give white crystalline Bempedoic acid (0.5 g).

### Example-10 h: Process for the Preparation of Crystalline Form of Bempedoic Acid

Bempedoic acid (1.0 g) was dissolved in butanone (10 mL), reaction mass was stirred at 50° C. for 3 hrs. Reaction mass was cooled to 25-30° C., the obtained solid was filtered and dried at 40° C. to give white crystalline Bempedoic acid (0.35 g).

We claim:

1. A compound of structure: 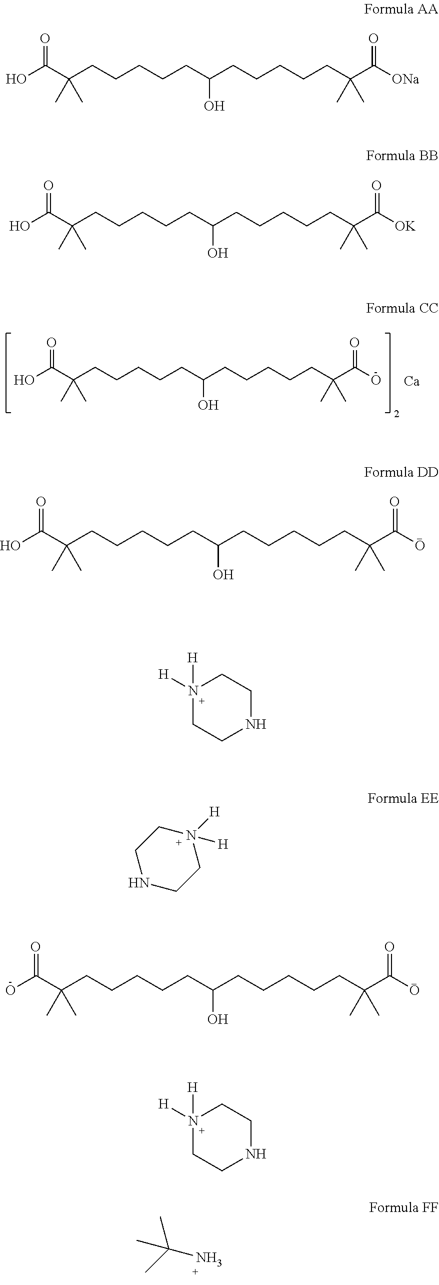 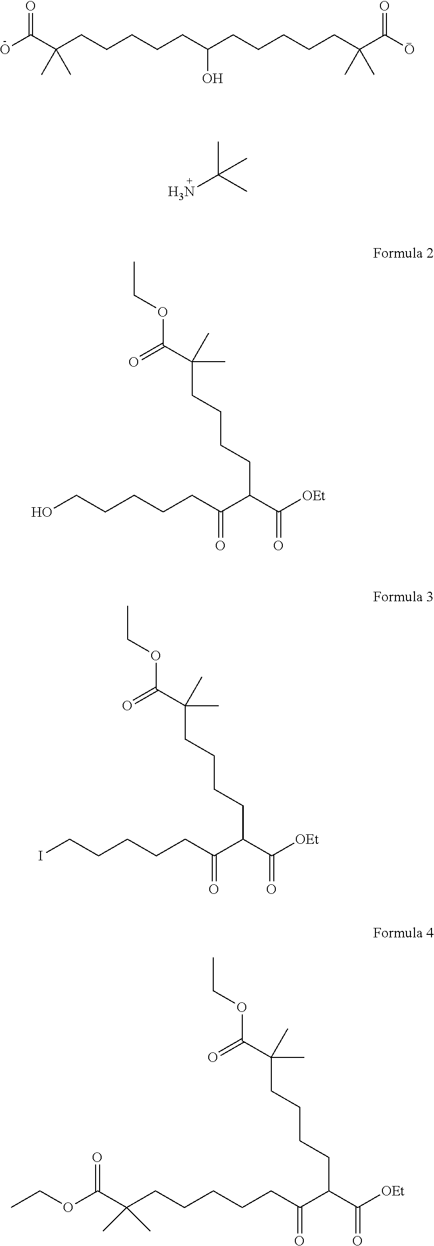 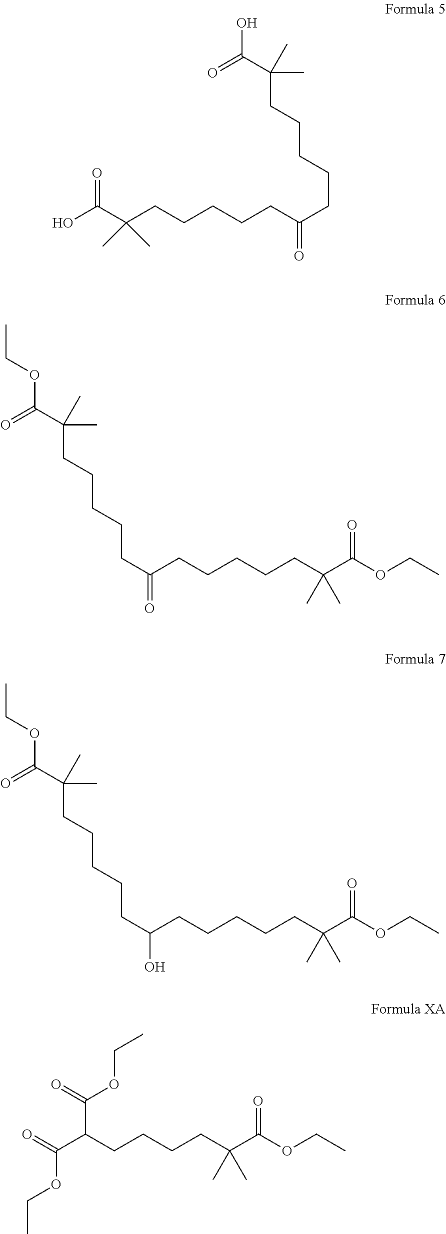 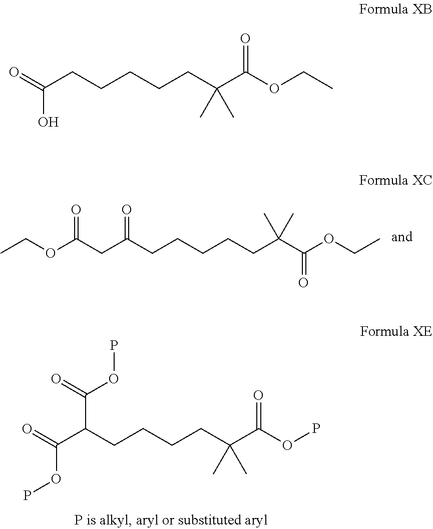
or a pharmaceutically acceptable salt, hydrate, or solvate thereof.

2-16. (canceled)

17. A process for the preparation of Bempedoic acid comprising the steps of:
i. treating diethyl-3-oxopentanedioate with ethyl 6-halo-2,2-dimethylhexanoate to give compound of Formula 4a; 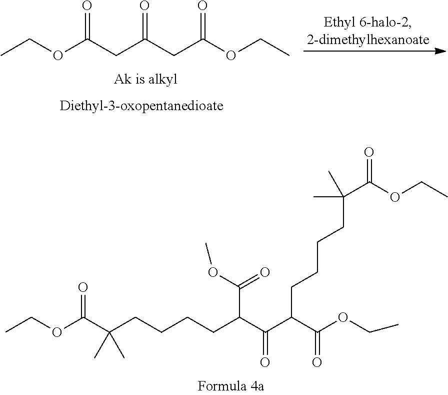
ii. treating compound of Formula 4a with a base to give compound of Formula 5 or compound of Formula 6; and 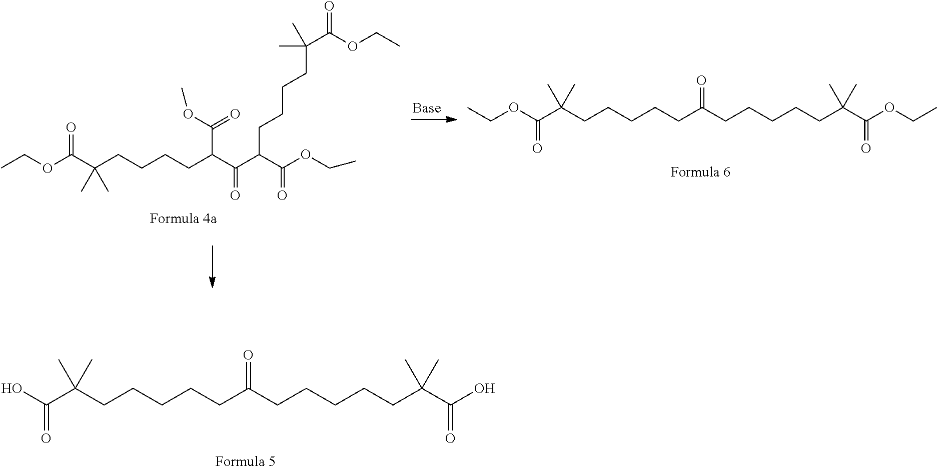 [SMILES: CCOC(=O)C(CCCCC(C)(C)C(=O)OCC)C(=O)C(CCCCC(C)(C)C(=O)OCC)C(=O)OC ; CCOC(=O)C(C)(C)CCCCCC(=O)CCCCCC(C)(C)C(=O)OCC ; CC(C)(CCCCCC(=O)CCCCCC(C)(C)C(=O)O)C(=O)O]
iii. converting isolated compounds of step ii (Formula 5 or Formula 6) to Bempedoic acid.

18. A process for the preparation of Bempedoic acid (Formula I) comprising the steps of:
a) treating ethyl isobutyrate with 1,5-dibromopentane in presence of base, potassium iodide to give compound of Formula 2a; 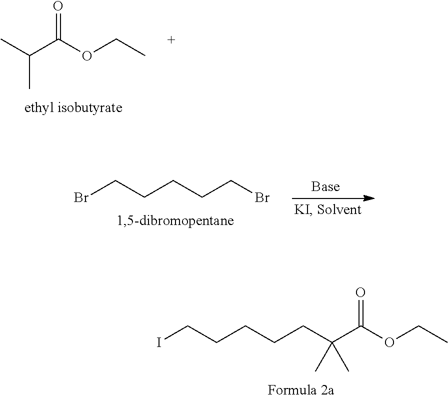
b) treating compound of Formula 2a with compound of Formula 2b in presence of a base to give compound of Formula 6; 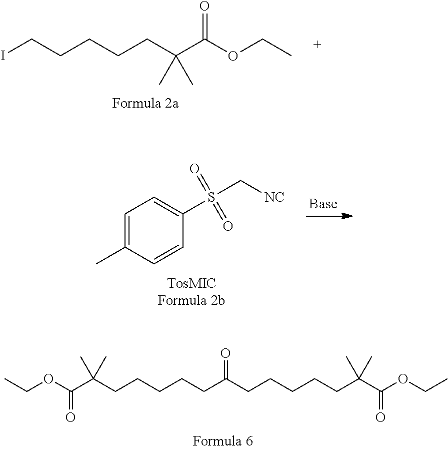
c) converting compound of formula 6 to compound of Formula 7; and 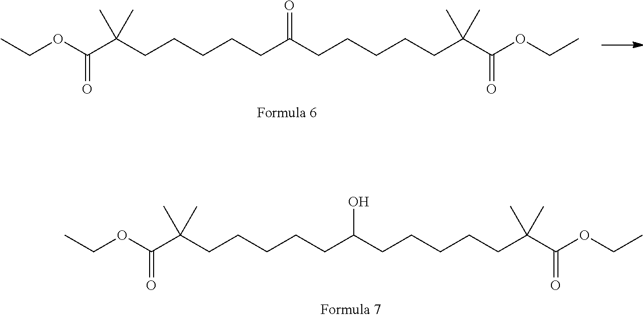
d) converting compound of formula 7 to Bempedoic acid (Formula I).

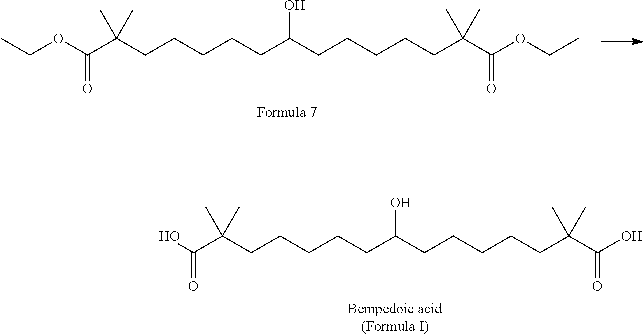 [SMILES: CCOC(=O)C(C)(C)CCCCCC(O)CCCCCC(C)(C)C(=O)OCC ; CC(C)(CCCCCC(O)CCCCCC(C)(C)C(=O)O)C(=O)O]

19. A process for the preparation of Bempedoic acid (Formula I) comprising the steps of:
a) treating caprolactone with ethyl acetate in presence of base to give ethyl 8-hydroxy-3-oxooctanoate; 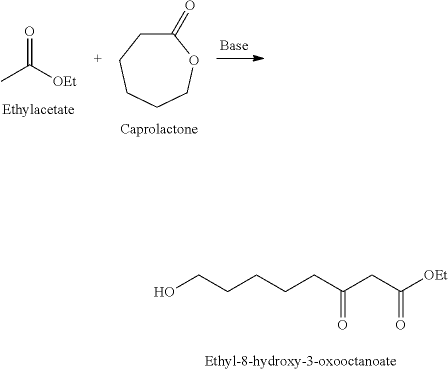
b) treating ethyl-8-hydroxy-3-oxooctanoate with ethyl 6-bromo-2,2-dimethylhexanoate to give diethyl 7-(6-hydroxyhexanoyl)-2,2-dimethyloctanedioate (Formula 2); 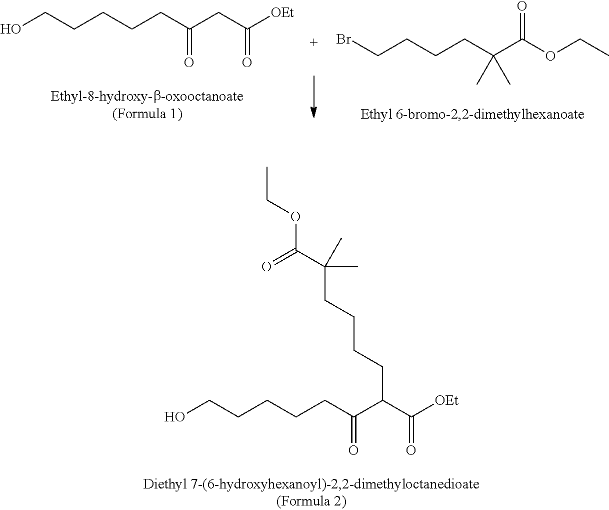 [SMILES: CCOC(=O)CC(=O)CCCCCO ; CCOC(=O)C(C)(C)CCCCBr ; CCOC(=O)C(CCCCC(C)(C)C(=O)OCC)C(=O)CCCCCO]
c) reacting diethyl 7-(6-hydroxyhexanoyl)-2,2-dimethyloctanedioate (Formula 2) with alkali metal halide or tetrabutylammonium halide to give diethyl 7-(6-hydroxyhexanoyl)-2,2-dimethyloctanedioate (Formula 3); 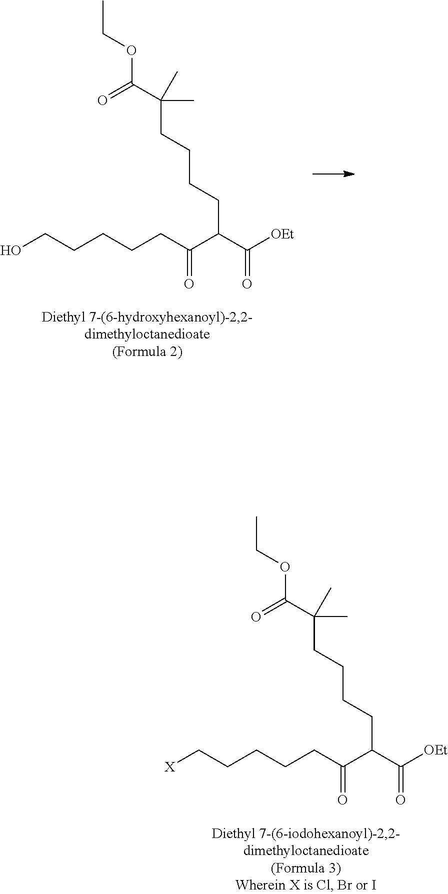
d) treating diethyl 7-(6-hydroxyhexanoyl)-2,2-dimethyloctanedioate (Formula 3) with ethyl isobutyrate in presence of a base to give triethyl 2,14-dimethyl-8-oxopentadecane-2,7,14-tricarboxylate (Formula 4); 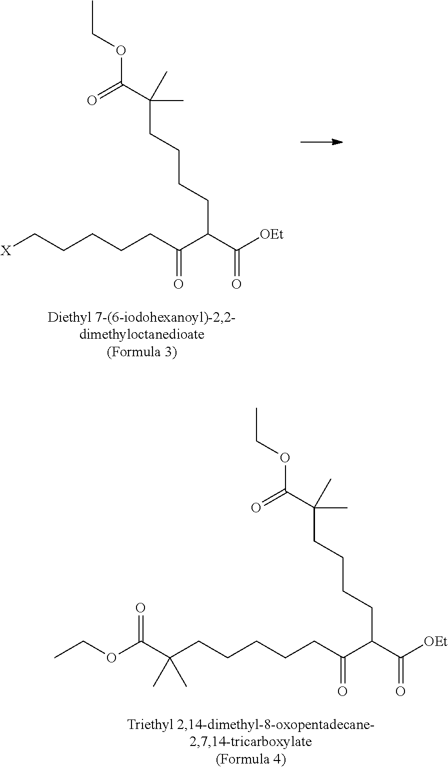
e) treating triethyl 2,14-dimethyl-8-oxopentadecane-2,7,14-tricarboxylate (Formula 4) with a base to give 2,14-dimethyl-8-oxopentadecanedioic acid (Formula 5); 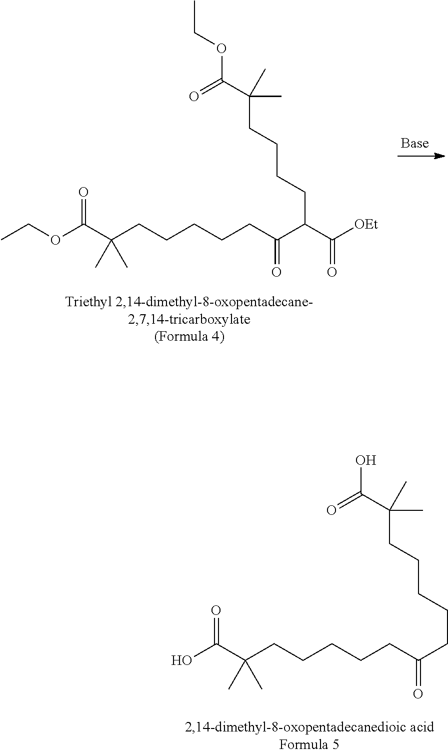 [SMILES: CCOC(=O)C(CCCCC(C)(C)C(=O)OCC)C(=O)CCCCCC(C)(C)C(=O)OCC ; CC(C)(CCCCCC(=O)CCCCCC(C)(C)C(=O)O)C(=O)O]
f) optionally treating, triethyl 2,14-dimethyl-8-oxopentadecane-2,7,14-tricarboxylate (Formula 4) with a base to give diethyl 2,2,14,14-tetramethyl-8-oxopentadecanedioate (Formula 6); 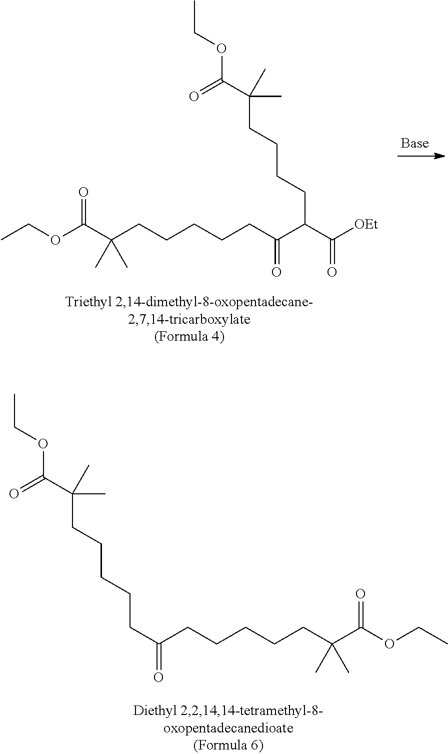 [SMILES: CCOC(=O)C(CCCCC(C)(C)C(=O)OCC)C(=O)CCCCCC(C)(C)C(=O)OCC ; CCOC(=O)C(C)(C)CCCCCC(=O)CCCCCC(C)(C)C(=O)OCC]
g) treating diethyl 2,2,14,14-tetramethyl-8-oxopentadecanedioate (Formula 6) with a reducing reagent to give diethyl 8-hydroxy-2,2,14,14-tetramethylpentadecanedioate (Formula 7); 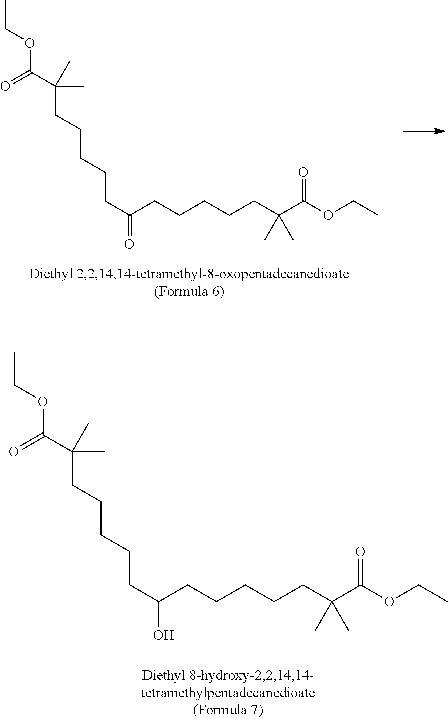 [SMILES: CCOC(=O)C(C)(C)CCCCCC(=O)CCCCCC(C)(C)C(=O)OCC ; CCOC(=O)C(C)(C)CCCCCC(O)CCCCCC(C)(C)C(=O)OCC]
h) optionally treating, 2,14-dimethhyl-8-oxopentadecanedioic acid (Formula 5) with a reducing reagent to give Bempedoic acid (Formula I); and
i) treating diethyl 8-hydroxy-2,2,14,14-tetramethylpentadecanedioate (Formula 7) with a base to give Bempedoic acid (Formula I).

20. A process for the preparation of diethyl 2,2,14,14-tetramethyl-8-oxopentadecanedioate comprising the steps of:
a) treating diethyl malonate with ethyl 6-bromo-2,2-dimethylhexanoatein presence of base to give compound of Formula XA; 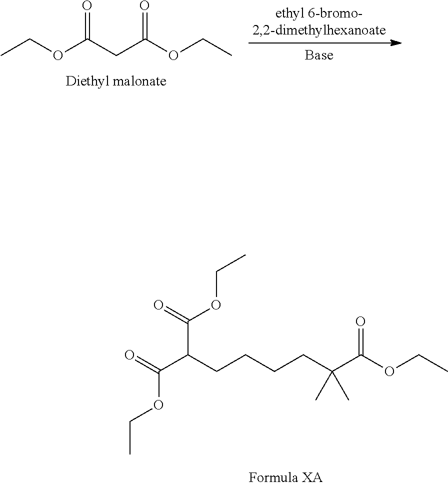 [SMILES: CCOC(=O)CC(=O)OCC ; CCOC(=O)C(CCCCC(C)(C)C(=O)OCC)C(=O)OCC]
b) treating compound of Formula XA with a base to give compound of compound of Formula XB; 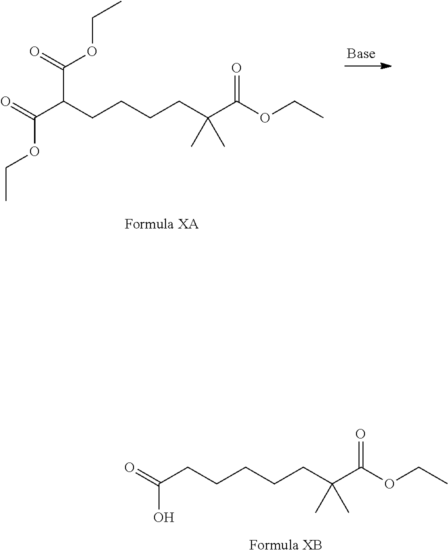 [SMILES: CCOC(=O)C(CCCCC(C)(C)C(=O)OCC)C(=O)OCC ; CCOC(=O)C(C)(C)CCCCCC(=O)O]
c) reacting compound of Formula XB with potassium 3-ethoxy-3-oxopropanoate to give compound of Formula XC; 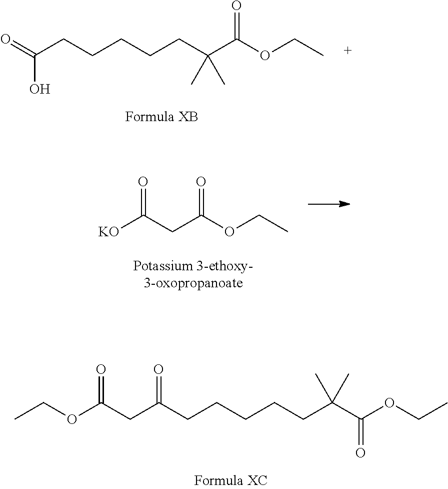
d) treating compound of Formula XC with ethyl 6-bromo-2,2-dimethylhexanote in presence of a base to give compound of Formula 4; 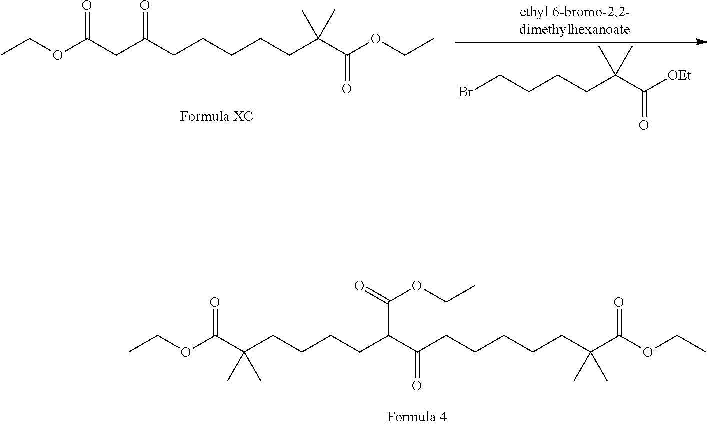
e) treating compound of formula 4 with a base to give diethyl 2,2,14,14-tetramethyl-8-oxopentadecanedioate; and
f) optionally, using diethyl 2,2,14,14-tetramethyl-8-oxopentadecanedioate intermediate to prepare Bempedoic acid.

21. A process for the preparation of crystalline Bempedoic acid comprising the steps of:
a) treating ethyl isobutyrate with 1,5-dibromopentane in presence of base, potassium iodide to give compound of formula 2a; 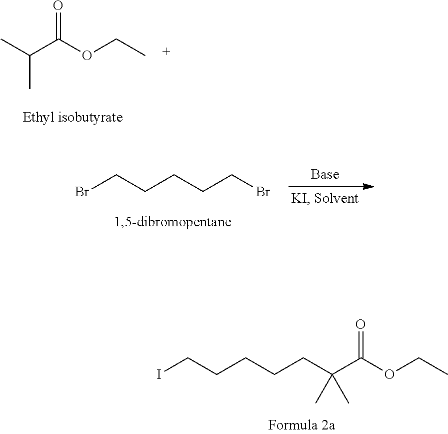
b) treating compound of formula 2a with compound of formula 2b in presence of a base to give compound of formula 6; 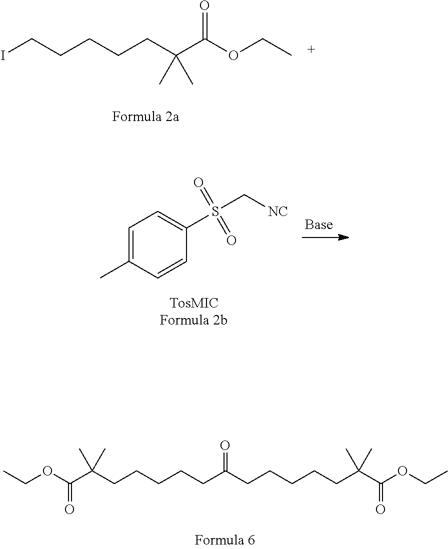
c) converting compound of formula 6 to compound of formula 7; and 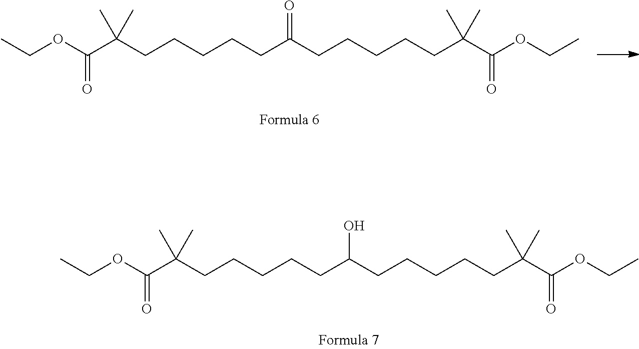
d) converting compound of formula 7 to crystalline Bempedoic acid.

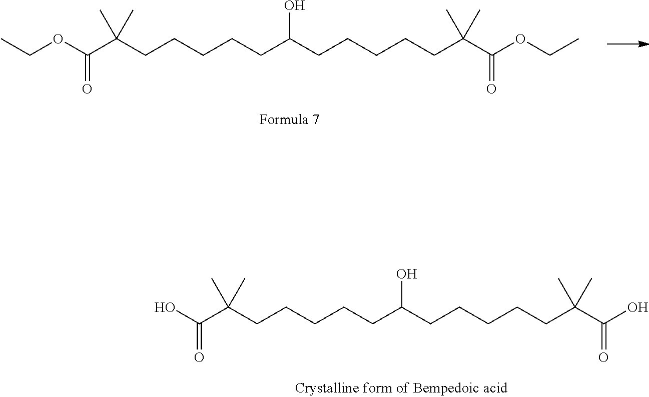 [SMILES: CCOC(=O)C(C)(C)CCCCCC(O)CCCCCC(C)(C)C(=O)OCC ; CC(C)(CCCCCC(O)CCCCCC(C)(C)C(=O)O)C(=O)O]

22. A process as claimed in claim 17, wherein the a) solvent is methanol, ethanol, acetone, methyl isobutyl ketone, ethyl methyl ketone, dichloromethane, chloroform, carbon tetrachloride, methyl acetate, ethyl acetate, n-propyl acetate, tetrahydrofuran, 2-methyl tetrahydrofuran, dimethyl ether, diethyl ether, diisopropyl ether, ethyl acetate, isopropyl acetate, tert-butylacetae, acetonitrile, Dimethyl sulfoxide, water or mixtures thereof; b) base is sodium hydride, potassium hydride, sodium butoxide, potassium butoxide, sodium methoxide, potassium methoxide, sodium ethoxide, potassium ethoxide, sodium propoxide, potassium propoxide, sodium beta-hydroxyethoxide, potassium beta-hydroxyethoxide, sodium hydroxide, potassium hydroxide, sodium oxide, potassium oxide, sodium carbonate, potassium carbonate, benzyl trimethylammonium methoxide, benzyl trimethylammonium hydroxide, methyl triphenylphosphonium methoxide, triphenylphosphonium hydroxide, triethylamine, N-methyl-di-isopropylamine, tri-n-butylamine, tri-n-octylamine or mixtures thereof; c) alkali metal iodide is sodium iodide or potassium Iodide or mixtures thereof; and d) reducing reagent is sodium borohydride, lithium borohydride, trimethoxy sodium boron hydride, tris ethyl lithium borohydride, lithium aluminum hydride, diisopropyl aluminum hydride, bis (2-methoxyethoxy) aluminum hydride or or mixtures thereof.

23. A process for the preparation of crystalline form of Bempedoic acid having PXRD characteristic peaks at 10.2°±0.2°, 17.4°+0.2°, 17.8°±0.2°, 18.6°±0.2°, 20.2°±0.2°, 21.7°±0.2°, 22.4°+0.2° and 23.4°±0.2° degrees 2θ, comprising the steps of:
a) dissolving Bempedoic acid in a solvent;
b) optionally adding second solvent, heating the reaction mass;
c) cooling the reaction mass; and
d) isolating crystalline form of Bempedoic acid.

24. A process as claimed in claim 23, wherein the solvent or second solvent is methanol, tetrahydrofuran (THF), acetone, ethyl acetate, isopropyl acetate, tertiary butyl acetate, acetonitrile, Dimethyl sulfoxide, di isopropyl ether, butanone or mixtures thereof.

25. A process as claimed in claim 18, wherein the a) solvent is methanol, ethanol, acetone, methyl isobutyl ketone, ethyl methyl ketone, dichloromethane, chloroform, carbon tetrachloride, methyl acetate, ethyl acetate, n-propyl acetate, tetrahydrofuran, 2-methyl tetrahydrofuran, dimethyl ether, diethyl ether, diisopropyl ether, ethyl acetate, isopropyl acetate, tert-butylacetae, acetonitrile, Dimethyl sulfoxide, water or mixtures thereof; b) base is sodium hydride, potassium hydride, sodium butoxide, potassium butoxide, sodium methoxide, potassium methoxide, sodium ethoxide, potassium ethoxide, sodium propoxide, potassium propoxide, sodium beta-hydroxyethoxide, potassium beta-hydroxyethoxide, sodium hydroxide, potassium hydroxide, sodium oxide, potassium oxide, sodium carbonate, potassium carbonate, benzyl trimethylammonium methoxide, benzyl trimethylammonium hydroxide, methyl triphenylphosphonium methoxide, triphenylphosphonium hydroxide, triethylamine, N-methyl-di-isopropylamine, tri-n-butylamine, tri-n-octylamine or mixtures thereof; c) alkali metal iodide is sodium iodide or potassium Iodide or mixtures thereof; and d) reducing reagent is sodium borohydride, lithium borohydride, trimethoxy sodium boron hydride, tris ethyl lithium borohydride, lithium aluminum hydride, diisopropyl aluminum hydride, bis (2-methoxyethoxy) aluminum hydride or or mixtures thereof.

26. A process as claimed in claim 19, wherein the a) solvent is methanol, ethanol, acetone, methyl isobutyl ketone, ethyl methyl ketone, dichloromethane, chloroform, carbon tetrachloride, methyl acetate, ethyl acetate, n-propyl acetate, tetrahydrofuran, 2-methyl tetrahydrofuran, dimethyl ether, diethyl ether, diisopropyl ether, ethyl acetate, isopropyl acetate, tert-butylacetae, acetonitrile, Dimethyl sulfoxide, water or mixtures thereof; b) base is sodium hydride, potassium hydride, sodium butoxide, potassium butoxide, sodium methoxide, potassium methoxide, sodium ethoxide, potassium ethoxide, sodium propoxide, potassium propoxide, sodium beta-hydroxyethoxide, potassium beta-hydroxyethoxide, sodium hydroxide, potassium hydroxide, sodium oxide, potassium oxide, sodium carbonate, potassium carbonate, benzyl trimethylammonium methoxide, benzyl trimethylammonium hydroxide, methyl triphenylphosphonium methoxide, triphenylphosphonium hydroxide, triethylamine, N-methyl-di-isopropylamine, tri-n-butylamine, tri-n-octylamine or mixtures thereof; c) alkali metal iodide is sodium iodide or potassium Iodide or mixtures thereof; and d) reducing reagent is sodium borohydride, lithium borohydride, trimethoxy sodium boron hydride, tris ethyl lithium borohydride, lithium aluminum hydride, diisopropyl aluminum hydride, bis (2-methoxyethoxy) aluminum hydride or or mixtures thereof.

27. A process as claimed in claim 20, wherein the a) solvent is methanol, ethanol, acetone, methyl isobutyl ketone, ethyl methyl ketone, dichloromethane, chloroform, carbon tetrachloride, methyl acetate, ethyl acetate, n-propyl acetate, tetrahydrofuran, 2-methyl tetrahydrofuran, dimethyl ether, diethyl ether, diisopropyl ether, ethyl acetate, isopropyl acetate, tert-butylacetae, acetonitrile, Dimethyl sulfoxide, water or mixtures thereof; b) base is sodium hydride, potassium hydride, sodium butoxide, potassium butoxide, sodium methoxide, potassium methoxide, sodium ethoxide, potassium ethoxide, sodium propoxide, potassium propoxide, sodium beta-hydroxyethoxide, potassium beta-hydroxyethoxide, sodium hydroxide, potassium hydroxide, sodium oxide, potassium oxide, sodium carbonate, potassium carbonate, benzyl trimethylammonium methoxide, benzyl trimethylammonium hydroxide, methyl triphenylphosphonium methoxide, triphenylphosphonium hydroxide, triethylamine, N-methyl-di-isopropylamine, tri-n-butylamine, tri-n-octylamine or mixtures thereof; c) alkali metal iodide is sodium iodide or potassium Iodide or mixtures thereof; and d) reducing reagent is sodium borohydride, lithium borohydride, trimethoxy sodium boron hydride, tris ethyl lithium borohydride, lithium aluminum hydride, diisopropyl aluminum hydride, bis (2-methoxyethoxy) aluminum hydride or or mixtures thereof.

28. A process as claimed in claim 21, wherein the a) solvent is methanol, ethanol, acetone, methyl isobutyl ketone, ethyl methyl ketone, dichloromethane, chloroform, carbon tetrachloride, methyl acetate, ethyl acetate, n-propyl acetate, tetrahydrofuran, 2-methyl tetrahydrofuran, dimethyl ether, diethyl ether, diisopropyl ether, ethyl acetate, isopropyl acetate, tert-butylacetae, acetonitrile, Dimethyl sulfoxide, water or mixtures thereof; b) base is sodium hydride, potassium hydride, sodium butoxide, potassium butoxide, sodium methoxide, potassium methoxide, sodium ethoxide, potassium ethoxide, sodium propoxide, potassium propoxide, sodium beta-hydroxyethoxide, potassium beta-hydroxyethoxide, sodium hydroxide, potassium hydroxide, sodium oxide, potassium oxide, sodium carbonate, potassium carbonate, benzyl trimethylammonium methoxide, benzyl trimethylammonium hydroxide, methyl triphenylphosphonium methoxide, triphenylphosphonium hydroxide, triethylamine, N-methyl-di-isopropylamine, tri-n-butylamine, tri-n-octylamine or mixtures thereof; c) alkali metal iodide is sodium iodide or potassium Iodide or mixtures thereof; and d) reducing reagent is sodium borohydride, lithium borohydride, trimethoxy sodium boron hydride, tris ethyl lithium borohydride, lithium aluminum hydride, diisopropyl aluminum hydride, bis (2-methoxyethoxy) aluminum hydride or or mixtures thereof.
